(19) United States
(12) Patent Application Publication
(10) Pub. No.: US 2025/0311971 A1
(43) Pub. Date: Oct. 9, 2025

(54) SOFT WIRELESS WEARABLE SENSOR SYSTEM AND METHOD FOR DETECTING SLEEP QUALITY AND DISORDERS

(71) Applicant: GEORGIA TECH RESEARCH CORPORATION, Atlanta, GA (US)

(72) Inventors: Woon-Hong Yeo, Atlanta, GA (US); Audrey Duarte, Atlanta, GA (US)

(21) Appl. No.: 18/873,169

(22) PCT Filed: Apr. 14, 2023
(86) PCT No.: PCT/US2023/018670

§ 371 Date: Dec. 9, 2024

Related U.S. Application Data
(60) Provisional application No. 63/350,609, filed on Jun. 9, 2022.
(60) Provisional application No. 63/404,377, filed on Sep. 7, 2022.

(51) Int. Cl. A61B 5/00; A61B 5/0205; A61B 5/257; A61B 5/291; A61B 5/296; A61B 5/297; A61B 5/372; A61B 5/398

(52) U.S. Cl. CPC A61B 5/4818; A61B 5/0205; A61B 5/257; A61B 5/291; A61B 5/296; A61B 5/297; A61B 5/372; A61B 5/398; A61B 5/4812; A61B 5/4815; A61B 5/6814; A61B 5/6823; A61B 5/7267

(57) ABSTRACT

Exemplary systems, methods, and devices are disclosed which provide at-home, portable, wireless sleep sensors and wearable electronics with embedded machine learning. Such devices have applications in assessing sleep quality and detecting sleep apnea with multiple patients. Unlike the conventional system at a sleep center using numerous bulky sensors, the soft all-integrated wearable platform offers natural sleep in a familiar setting. The face-mounted patches that detect brain, eye, and muscle signals show comparable performance in sleep monitoring with polysomnography in a clinical study. Furthermore, deep learning is embedded in an exemplary device, offering automated high-precision sleep scoring, which demonstrates the wearable system's portability and point-of-care usability. The at-home wearable patches help to support portable sleep monitoring and home healthcare.

### RELATED APPLICATION

This application claims priority to, and the benefit of, U.S. Provisional Patent Application No. 63/350,609, filed Jun. 9, 2022, “entitled “WEARABLE, NEURO-BIOPATCH FOR MONITORING OF SLEEP TIME AND QUALITY” and U.S. Provisional Patent Application No. 63/404,377, filed Sep. 7, 2022, entitled “WEARABLE, NEURO-BIOPATCH FOR MONITORING OF SLEEP TIME AND QUALITY,” each of which is hereby incorporated by reference herein in its entirety.

### STATEMENT REGARDING FEDERALLY SPONSORED RESEARCH OR DEVELOPMENT

This invention was made with government support under Award No. R21AG064309 awarded by the National Institutes of Health. The government has certain rights in the invention.

### BACKGROUND

Sleep disorder is a prevalent issue. Although many people suffer from sleep disorders, most are undiagnosed, leading to impairments in health and daily functioning and a massive economic burden.

Current technique to diagnose sleep disorder involves a single night polysomnography (PSG) test. Diagnosis can ultimately guide months or years of long-term treatment. PSG is resource-intensive, requiring a specialized laboratory and equipment, 20+ wired sensor leads, and trained technician staff to set up the study and score the data before a physician can provide a diagnosis. Because of its complexity, polysomnography can typically only be conducted for a single, discrete night in high-resource clinical settings. Therefore, the existing sleep monitoring methods using polysomnography is not easily accessible, costly, and burdensome to patients, requiring specialized facilities and trained personnel. While home sleep apnea testing devices with reduced numbers of sensors do exist, they still employ many wires and are targeted to measuring body movements, blood oxygen saturation, and such.

Thus, there is a benefit and/or a need to improve systems and methods for detecting sleep quality and disorders.

### SUMMARY

Exemplary systems, methods, and devices are disclosed for an at-home, portable, wireless sleep sensors and wearable electronic device having a reduced sensor set that may be employed in combination with embedded machine learning that can provide sleep stage classification and/or apnea event detection. The exemplary system and method may have applications/utility in assessing sleep quality and detecting sleep apnea. Unlike the conventional system at a sleep center using numerous bulky sensors, the exemplary system and method employs soft all-integrated wearable platform that can be used in natural sleep in a familiar setting for a patient. The soft all-integrated wearable platform includes a set of one or more face-mounted patches that can detect brain, eye, and muscle signals. The exemplary system and method, in employing reduced sensor set, is observed to beneficially have comparable performance in sleep monitoring with polysomnography in a clinical setting. In fact, in a study, when comparing healthy controls to sleep apnea patients, the exemplary wearable system was observed to be able to detect obstructive sleep apnea events with an accuracy of 88.5%, the highest to date. The deep learning embedded in the exemplary device further provides an automated high-precision sleep scoring, which demonstrates the wearable system's portability and point-of-care usability. The at-home wearable patches help to support portable sleep monitoring and home healthcare.

A clinical study involving sleep patients and healthy controls was performed, and it fully validated the wearable device's performance. In one implementation, a portable platform was fabricated having only two unobtrusive patches for clinical-grade sleep analysis that use Bluetooth short-range communication to transfer data to a tablet or smartphone wirelessly. Unlike the traditional PSG devices, which may employ ten wired sensors and bulky electronics, the prototyped wearable patch could be employed anywhere, like a user's home, to offer a natural and comfortable sleep. Collectively, the wireless wearable biomedical systems can be combined with machine learning technologies to provide home-sleep monitoring platform, as well as clinical platform, for home healthcare, digital health monitoring, and quantitative disease diagnosis.

In one aspect, an exemplary wearable biopatch device configured for sleep monitoring is disclosed. The device includes a hypoallergenic silicone adhesive substrate comprising an inner surface and an outer surface. The device further includes an integrated sensor system coupled to the inner surface of the silicone adhesive substrate. The integrated sensor system includes a plurality of electrodes placed in predetermined locations and electrical connections between the plurality of electrodes, including at least three electrodes configured for EEG sensing and at least three electrodes for EOG sensing. The device further includes a silicone fabric substrate disposed on the outer surface of the silicone adhesive substrate. The hypoallergenic silicone adhesive substrate and silicone fabric substrate provide conformal and stretchable substrate for conformal and stretchable contact on a facial area. The device further includes at least one circuit configured for wireless data transmission disposed on a portion of the silicone fabric substrate, the at least one circuit comprising thin, flexible film connectors.

In some implementations, the wearable biopatch device is attachable a forehead portion of the facial area and configured to measure EEG and EOG. The device further includes a second wearable biopatch attachable to the chin (e.g., attachable above a mandible line to sit on the chin in a sleep position) and configured to measure EMG. The second wearable biopatch is electrically linked to the wearable biopatch device to provide synchronous measurements of the EEG sensing, EOG sensing, and EMG sensing.

In some implementations, the device is configured to monitor brain activity and eye movement to provide the EEG sensing and EOG sensing to an analysis system configured with a trained machine learning or neural network to provide sleep stage classification and apnea event detection. The trained machine learning or neural network is configured to provide output for monitoring, tracking, and/or diagnose obstructive sleep apnea.

In some implementations, the device is configured to monitor brain activity, eye movement, and facial muscle activity signals EEG sensing, EOG sensing, and EMG sensing to an analysis system configured with a trained machine learning or neural network to provide sleep stage classification and apnea event detection. The trained machine learning or neural network is configured to provide output for monitoring, tracking, and/or diagnose obstructive sleep apnea.

In some implementations, a portion of the plurality of electrodes are stretchable and formed in a meandering or serpentine structure (e.g., gold membrane electrodes or graphene electrodes formed by laser micromachining). In some implementations, all of the plurality of electrodes are stretchable and formed in a meandering or serpentine structure.

In some implementations, the at least three electrodes configured for EEG sensing includes a common ground electrode, a common reference electrode, and a first recording electrode. The at least three electrodes configured for EOG sensing includes a second recording electrode, the common ground electrode, and the common reference electrode.

In some implementations, the at least one circuit further comprises: a multi-channel differential amplifier; a Bluetooth-low-energy microcontroller; and an antenna (e.g., a 2.4 GHz antenna).

In some implementations, the silicone fabric substrate includes polytetrafluoroethylene (PTFE). In some implementations, the hypoallergenic silicone adhesive substrate is between 50 micrometers and 300 micrometers thick (e.g., 250 micrometers thick).

In some implementations, the device further includes a chest-mounted cardiorespiratory patch.

In some implementations, the device further includes a remote analysis system (e.g., wireless monitoring system) separate from the wearable biopatch device. The remote analysis system is configured to receive signals from the wearable biopatch device and either (i) relay the received signals to a cloud analysis system having the trained machine learning or neural network or (ii) perform the analysis with a locally executing trained machine learning or neural network.

In some implementations, the remote analysis system or the cloud analysis system is configured to provide a sleep score associated with the sleep stage classification and apnea event detection.

In another aspect, a system is disclosed, the system including the wearable biopatch device herein described. The system further includes an analysis system separate from the wearable biopatch device. The remote analysis system is configured to receive signals from the wearable biopatch device and either (i) relay the received signals to a cloud analysis system having the trained machine learning or neural network or (ii) perform the analysis with a locally executing trained machine learning or neural network.

In some implementations, the analysis system comprises cloud infrastructure. In some implementations, the analysis system comprises a smart phone, a tablet, or any other personal computing device having a user interface configured to display at least one of the transmitted data and the sleep score and configured with a short-range communication interface.

In another aspect, a method of sleep monitoring is disclosed, the method including: (i) providing a wearable biopatch device. The wearable biopatch device includes: a hypoallergenic silicone adhesive substrate comprising an inner surface and an outer surface; an integrated sensor system coupled to the inner surface of the silicone adhesive substrate, the integrated sensor system comprising a plurality of electrodes placed in predetermined locations and electrical connections between the plurality of electrodes, including at least three electrodes configured for EEG sensing and at least three electrodes for EOG sensing; a silicone fabric substrate disposed on the outer surface of the silicone adhesive substrate, the hypoallergenic silicone adhesive substrate and silicone fabric substrate providing conformal and stretchable substrate for conformal and stretchable contact on a facial area; and at least one circuit configured for wireless data transmission disposed on a portion of the silicone fabric substrate, the at least one circuit comprising thin, flexible film connectors. The method further includes: (ii) sensing, via the plurality of electrodes, EEG signals from the at least three electrodes configured for EEG sensing and EOG signals from the at least three electrodes configured for EOG sensing over a period spanning at least one REM cycle; (iii) transmitting the sensed EEG signals and the sensed EOG signals to local computing device; (iv) processing and segmenting, at the local computing device, the sensed EEG signals and the sensed EOG signals; (v) analyzing, via the local computing device or a remote analysis system that received the data from the local computing device, the segmented EOG signals and the segment EEG signals via a trained machine learning operation (e.g., convolutional neural networks (CNN)); and (vi) providing a sleep score associated with a sleep disorder to a graphical user interface (e.g., associated with the local computing device) based on the analyzed brain activity data.

In some implementations, the method further includes: classifying, based on the sleep score, a sleep stage; and detecting, based on the sleep score, a sleep apnea event.

In some implementations, the at least three electrodes associated with the EEG sensing are placed on the forehead to acquire the EEG signals, and the at least three electrodes associated with the EOG sensing are placed at the temple to acquire the EOG signals.

In some implementations, the wearable biopatch device is self-applying to be installed by the subject, and wherein the EEG signals and EOG signals are monitored by a user at a remote location.

### BRIEF DESCRIPTION OF DRAWINGS

The skilled person in the art will understand that the drawings described below are for illustration purposes only.

FIG. 1 is a diagram of an exemplary wearable biopatch device, according to one implementation.

FIGS. 2A, 2B, 2C, 2D, and 2E show additional diagrams of the example wearable biopatch device of FIG. 1.

FIG. 3 shows details of an example multi-layered flexible circuit of the device of FIG. 1, according to one implementation.

FIG. 4 shows an example analysis system utilizing the device of FIG. 1, according to one implementation.

FIG. 5A shows one example of the architecture of a wearable biopatch device.

FIG. 5B shows a diagram of the circuitry, operation, and analysis of the exemplary wearable biopatch device of FIG. 5A, according to one implementation.

FIG. 6A shows images of a fabricated array of stretchable gold electrodes for the exemplary wearable biopatch device, according to one implementation.

FIG. 6B shows an image of fabricated copper stretchable interconnectors and a corresponding microscopic image of the interconnectors, according to one implementation.

FIG. 6C shows an image of an example electrode with interconnector on a fabric surface, according to one implementation.

FIG. 6D shows an image of an example electrode with interconnector disposed on a curved surface along with an optical microscope image of the fabric fibers, according to one implementation.

FIG. 6E shows a cross-sectional optical microscope image of the fiber of FIG. 6D, according to one implementation.

FIG. 6F (having subpanels A and B) shows images of stretching a circuit with and without a strain isolation layer, according to one implementation.

FIG. 6G shows the method of fabrication and assembly of the components of the example wearable biopatch device.

FIG. 6H shows diagrams of the circuit component of the example wearable biopatch device, according to one implementation.

FIGS. 7A and 7B show finite element analysis (FEA) of the mechanical stretchability features of the stretchable interconnectors and stretchable electrodes of FIGS. 3A and 3B, according to one implementation.

FIGS. 7C, 7D, and 7E show mechanical testing results of the stretchable interconnectors and stretchable electrodes. Specifically, FIGS. 7C shows images of a mechanical reliability test of fabricated interconnectors and stretchable electrodes in a stretching test, according to one implementation. FIG. 7D is a plot of the measured cyclic stretching characterization of the electrode and connector, according to one implementation. FIG. 7E is a plot of the thickness and peeling strength characterization of the electrode and connector, according to one implementation.

FIG. 7F is a plot showing the mechanical and electrical properties of the example wearable biopatch device over an extended period. The plot includes measured peeling strength values and signal-to-noise ratio (SNR) of measured contact signals with the skin for seven days of sleep, according to one implementation.

FIGS. 8A, 8B, 8C, and 8D show scanning electron microscope (SEM) images of various membrane materials and thicknesses showing examples of skin-electrode contact quality.

FIG. 8E shows a graph of an analytical calculation of total interface energy with varying thicknesses of Silbione, according to one implementation.

FIG. 8F shows a diagram showing the relationship between work of adhesion, modulus, and resultant conformal skin contact for various materials, according to one implementation.

FIG. 8G shows a graph of impedance density for various materials, according to one implementation.

FIGS. 9A, 9B, 9C, 9D, and 9E show mechanical/electrical failure evaluations of the electrode and connector. Specifically, FIG. 9A (having subpanels A and B) shows additional finite element analysis (FEA) of the stretchability features of the electrode and connector in mechanical/electrical failure evaluations, according to one implementation. FIGS. 9B-9C show images of a gold electrode, copper connector, and combined system before and after mechanical/electrical failure evaluation, according to one implementation. FIG. 9D (panels a, b, and c) show graphs illustrating the resistance change of a gold electrode, copper connector, and combined system, according to one implementation. FIG. 9E shows a graph of the strain-stress curves of fabric stretched in three different directions up to failure, according to one implementation.

FIG. 10A (having subpanels A, B, C, D, E, and F) shows an array of images of fabric substrates, according to various implementation. FIG. 10B (having panels A, B, C, and D) shows an image of a test setup along with curve fit lines and their work of adhesion values for three materials, according to various implementations.

FIGS. 11A, 11B, 11C, 11D, 11E, 11F, and 11G shows results of a comparative study between the example wearable biopatch device and a conventional PSG device. Specifically, FIG. 11A shows an image of a subject in a sleep clinic wearing the two different sleep systems. FIG. 11B shows graphs of physiological signals of the two different sleep systems measured during five sleep stages. FIGS. 11C and 11D show spectrogram sleep data of the two different sleep systems. FIG. 11E and 11F present two representative sleep scoring analysis of the two different sleep systems. FIG. 11G shows a confusion matrix summarizing comparison results of the two different sleep systems acquired from eight subjects detecting five sleep stages, according to various implementations.

FIGS. 12A, 12B, 12C shows raw signal data acquired by the example wearable biopatch device. Specifically, FIG. 12A (having subpanels A, B, C, D, and E) shows graphs of raw sleep data measured by the example wearable patch with all five channels during five sleep stages. FIG. 12B (having subpanels A, B, C, and D) shows graphs of the spectral power value comparison of EEG signals from PSG and an example system. FIG. 12C (having subpanels A and B) shows spectrogram data of seven consecutive sleep measurements generated from EEG1 signals at each day of seven consecutive sleep measurements.

FIGS. 13A, 13B, and 13C show results of the sleep characterization. FIG. 13A (having subpanels A, B and C) shows an EEG hypnogram and a spectrogram measured from a healthy control using a wireless wearable patch, according to one implementation. FIG. 13B (having subpanels A, B, and C) shows a hypnogram capturing the numerous arousals following series of apnea and a corresponding spectrogram depicting the characteristics of fragmented sleep signals, according to one implementation. FIG. 13C (having subpanels A, B, and C) provides a comparison of single-and multi-taper spectrogram data, according to one implementation.

FIGS. 14A, 14B, 14C, 14D, 14E, 14F, 14G show an example deep learning system and its characterization. Specifically, FIG. 14A shows diagrams of a deep learning system from data acquisition to training, processing, and prediction, according to one implementation. FIG. 14B shows the deep learning architecture developed in a study that uses sleep data measured by the wearable patch for automated sleep scoring, according to one implementation. FIGS. 14C-14D show graphs of representative results of the automated sleep scoring, according to various implementations. FIG. 14E provides a confusion matrix summarizing the performance of a CNN algorithm compared to manual scoring with PSG, according to one implementation. FIG. 14F provides a set of diagrams depicting the input data and the CNN architecture for sleep apnea detection, according to one implementation. FIG. 14G provides an analysis of the confusion matrix of FIG. 14E.

FIGS. 15A and 15B show example CNN-based architecture for sleep stage detection and/or sleep apnea detection. Specifically, FIG. 15A provides a flowchart with details of the CNN algorithm, data processing, and classification processes for sleep stage detection and sleep apnea detection, according to one implementation. FIG. 15B provides a flowchart with information of the CNN+GRU architecture, showing a machine learning architecture of another deep neural network for sleep apnea detection, according to one implementation.

FIGS. 16A, 16B, 16C, 16D, 16E, 16F, 16G, 16H, 16I, 16K, and 16L show two prototyped iterations of the example wearable biopatch device. FIGS. 16A-16C show an iteration of the forehead sensor. FIGS. 16D-16F show an iteration of the chin sensor. FIGS. 16G-16I show another iteration of the forehead sensor. FIGS. 16J-16L show another iteration of the chin sensor.

### DETAILED DESCRIPTION

Each and every feature described herein, and each and every combination of two or more of such features, is included within the scope of the present disclosure, provided that the features included in such a combination are not mutually inconsistent.

As discussed above, a fully portable and highly skin-conformable at-home sleep monitoring system is presented that integrates soft and functional materials with electronics for a comfortable yet reliable wearable system. This wireless wearable platform addresses the existing challenges and limitations of the gold-standard sleep monitoring tools and methods used at sleep clinics. Soft hybrid manufacturing and packaging technologies may be utilized to offer enhanced mechanical reliability and comfortable wearability with conformal device lamination to the skin. Laser micromachining may be used for scalable manufacturing of nanomembrane stretchable sensors and interconnectors. A composite of elastic fabric and ultrasoft silicone elastomer makes a substrate to integrate sensors and electronics together, providing strain distribution and strong adhesion to the skin. The soft wearable platform, mounted on the face, wirelessly measures high-quality sleep physiological signals, including EEG (electroencephalogram), EOG (electrooculography), and EMG (electromyography), which are comparable to the data recorded by the PSG system at a sleep clinic. In addition, a deep-learning algorithm may be implemented (e.g., convolutional neural networks (CNN)). When the CNN is embedded in the portable sleep patches, automated quantitative sleep scoring and apnea detection is provided.

### Example System

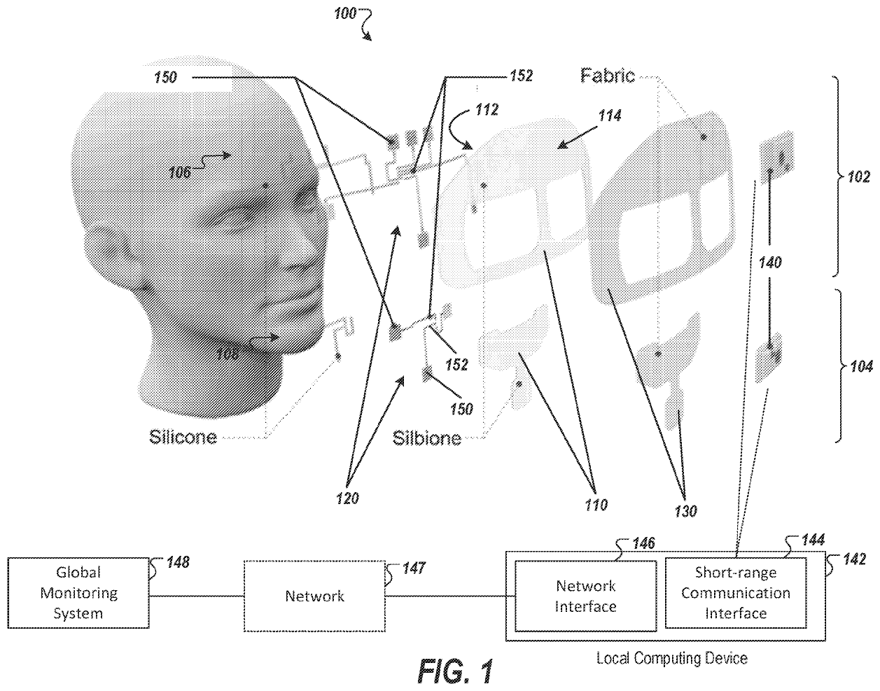

FIG. 1 present an example overview of an at-home sleep monitoring patch (e.g., a wearable biopatch device 100). The portable, wearable biopatch device 100 includes two small patches: one for measuring EEG and EOG on the forehead (shown as forehead patch 102) and the other for measuring EMG on the chin (shown as chin patch 104). In some implementations, the device 100 includes an additional patch device on a different portion of the body of a user (e.g., chest-mounted cardiorespiratory patch).

In the diagram shown in FIG. 1, the exploded view of the wearable biopatch device 100 is shown. The wearable biopatch device, or simply “device” 100, for the forehead and chin sensors, includes a hypoallergenic silicone adhesive substrate 110, stretchable sensor components 120 having the stretchable electrodes and interconnects, a silicone fabric substrate 130, and at least one controller and communication circuit 140. The soft and unobtrusive patch has an exceptionally smaller form factor than other wearable sleep monitors, offering seamless integration with the skin for high-fidelity, reliable signal detection during sleep as later described herein.

The device includes a hypoallergenic silicone adhesive substrate 110, or simply “adhesive substrate 110” having an inner surface 112 and an outer surface 114. The silicone adhesive substrate can be 250 micrometers thick. In other implementations the silicone adhesive substrate is 50 micrometers or more. In other implementations, the silicone adhesive substrate is 300 micrometers thick or less.

The stretchable sensor components 120 are coupled to the inner surface 112 of the silicone adhesive substrate 110 and includes a plurality of electrodes 150 disposed in predetermined locations and electrical connections 152 between the plurality of electrodes 150 (e.g., nanomembrane electrodes and stretchable copper interconnectors). At least three electrodes 150 are configured for EEG (electroencephalogram) sensing, and at least three electrodes 150 are configured for EOG (electrooculography) sensing (which may be inclusive of one or more of the same electrodes 150 used for EEG sensing).

All electronic components of device 100 are embedded in a soft fabric composite made of elastic non-woven polyurethane and medical-grade silicone adhesive. The fabric packaging provides a non-sticky dry surface for convenient device handling while protecting the electronics from excessive mechanical deformation [26, 27]. The dry electrodes 150 on a patterned polyimide film offer reusability for multiple days of sleep recording, unlike the one-time-use gel electrodes in the standard PSG [28-30]. In other implementations, other fabric, plastic, and adhesive materials may be used.

The silicone fabric substrate 130 is disposed on the outer surface 114 of the silicone adhesive substrate 110, the silicon adhesive substrate 110 and silicone fabric substrate 130 providing conformal and stretchable substrate for conformal and stretchable contact on a facial area. The silicone fabric substrate 130 includes polytetrafluoroethylene (PTFE), but, in other implementations, the silicone fabric substrate includes any flexible fabric material or flexible plastic material.

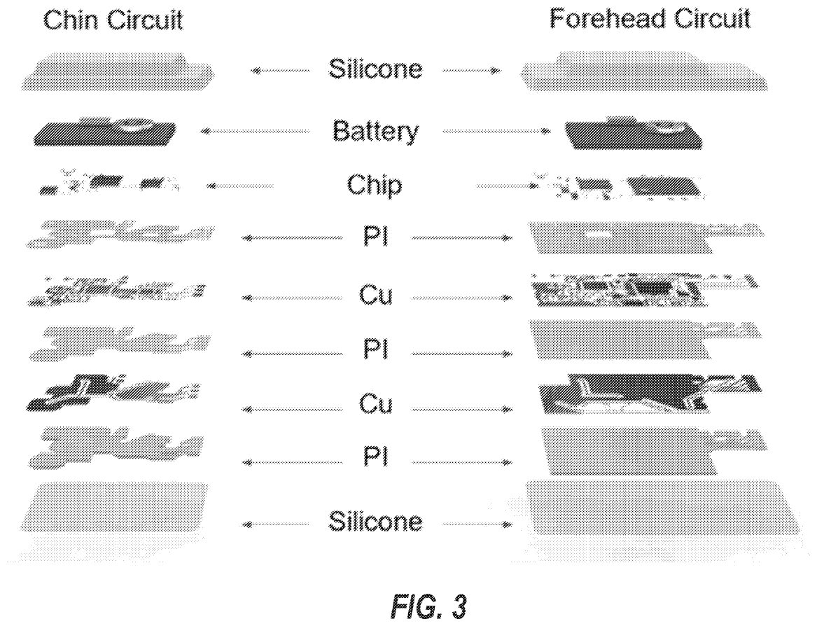

The control and communication circuit 140 acquires the signals from the sensors and provide wireless data transmission of the acquired signals to a local computing device 142. The circuit 140 is shown disposed on a portion of the silicone fabric substrate 130. The circuit 140 is formed a flexible film that couples, through thin, flexible film connectors (e.g., a stretchable copper interconnector encapsulated with an elastomer), to the stretchable sensor components 120. The local computing device 142 includes a short-range communication interface 144 (to interface with the control and communication circuit 140) and a network interface 146 (to interface through a network 147 to a global monitoring system 148). FIG. 3 shows details of an example multi-layered flexible circuit 140, made of metals, polymers, and chips, which are fully encapsulated by silicone membranes for strain isolation during device assembly, handling, and wearing during sleep.

The local computing device 142 may be a smart phone, personal computer, or edge computing device (provided by the healthcare provider) that can relay the acquired signals from the sensors to the global monitoring system 148. The network 147 may be a wide-area-network (e.g., for at-home monitoring) or local-area-network (e.g., for hospital or clinic).

The global monitoring system 148 is a set of back-end infrastructure (e.g., cloud infrastructure) that provides monitoring of multiple devices (e.g., 100). The global monitoring system 148 may include one or more analysis engine or systems configured to perform the sleep classification or apnea detection using the algorithms described herein. The global monitoring system 148 may provide an interface/dashboard that allows a single technician to monitor the sleep session of individual patients or subjects.

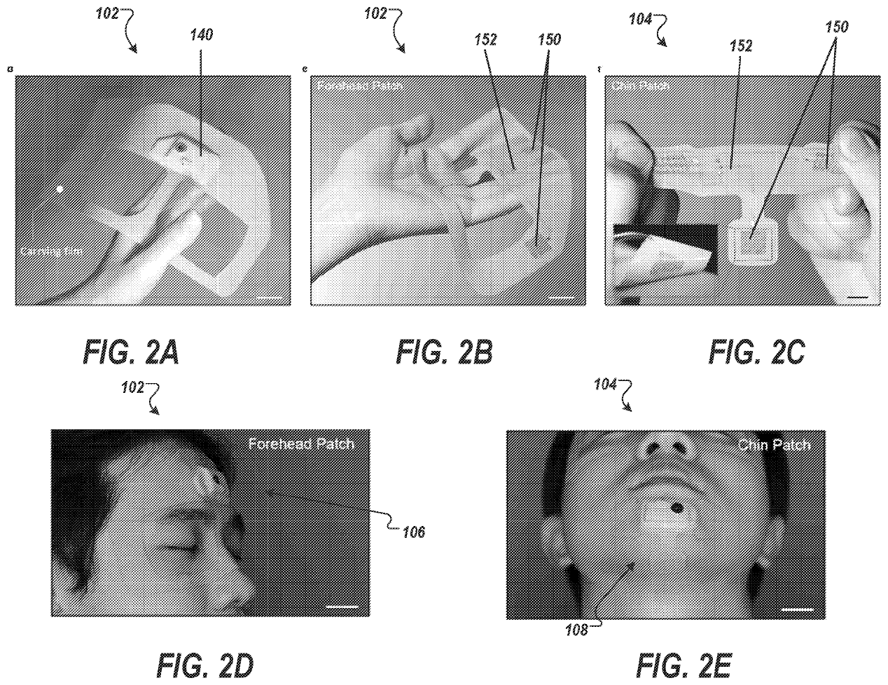

FIGS. 2A, 2B, 2C, 2D, and 2E show additional diagrams of the example wearable biopatch device of FIG. 1. Specifically, FIGS. 2A-2B show examples of assembled devices 100 with FIGS. 2C and 2E showing the second wearable biopatch or chin patch 104 on the chin 108. The forehead patch 102 of the device 100 is attachable to a forehead portion 106 of the facial area and configured to measure EEG and EOG. In some implementations, the forehead patch 102 the device 100 is sufficient for capturing desired sleep signals. However, the device 100 of FIG. 1 further includes a second wearable biopatch 104 (e.g., chin patch 104) attachable to the chin 108 (e.g., attachable above a mandible line to sit on the chin in a sleep position).

The chin patch 104 is configured to measure EMG (electromyography). The second wearable biopatch 104 is electrically linked to the forehead patch 102 of the wearable biopatch device 100 to provide synchronous measurements of the EEG sensing, EOG sensing, and EMG sensing.

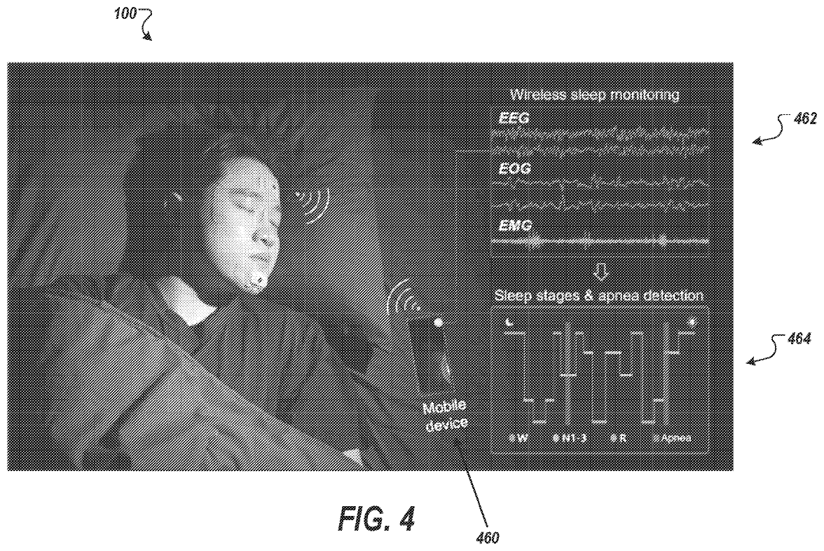

The device 100 is configured to monitor brain activity and eye movement to provide the EEG sensing and EOG sensing to an analysis system configured with a trained machine learning or neural network to provide sleep stage classification and apnea event detection. The device 100 is also configured to monitor brain activity, eye movement, and facial muscle activity signals EEG sensing, EOG sensing, and EMG sensing to an analysis system. The trained machine learning or neural network is configured to provide output for monitoring, tracking, and/or diagnose obstructive sleep apnea. FIG. 4 shows an example analysis system in which the device 100 communicates with a mobile device 460 which can then receive the EEG, EOG, and EMG signals, shown in exemplary graph 462. The mobile device 460 (or some other processing unit) can provide the sleep stage classification and apnea event detection as shown in exemplary graph 464.

### Example Control System and Electronics

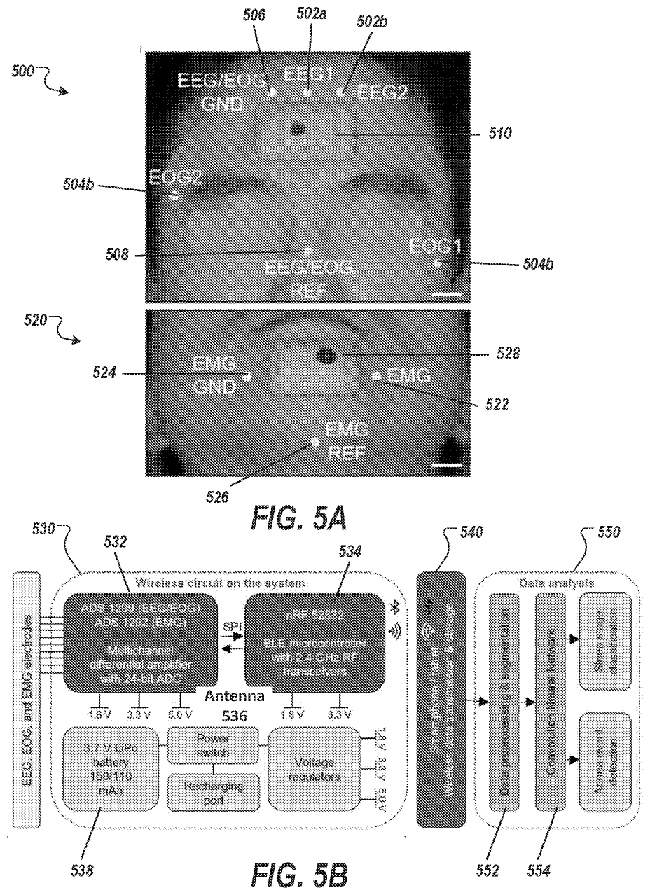

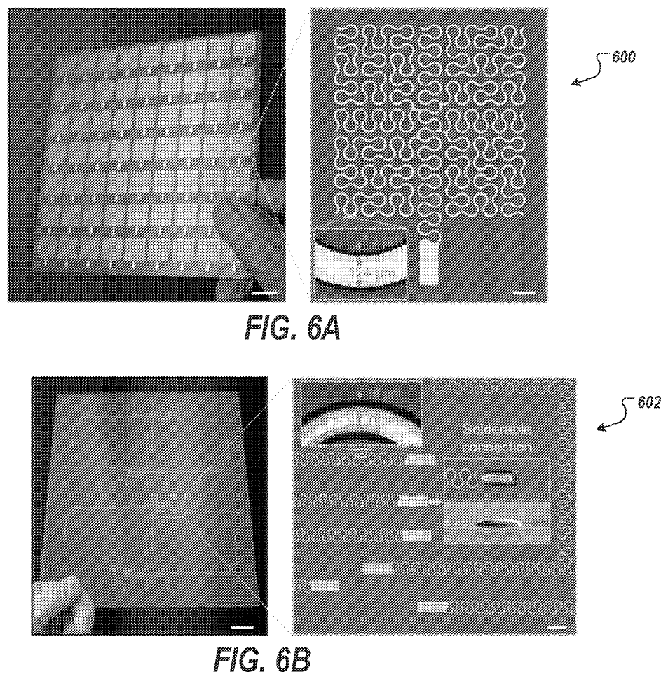

FIGS. 5A and 5B show one example of the architecture of the control system and electronics of the device 500. FIG. 5A shows the forehead patch 502 attached to a forehead portion and a chin patch 520 attached to a chin (e.g., similar to device 100 with forehead patch 102 and chin path 104 in FIG. 1). Each of the forehead patch 500 and the chin patch 520 includes the stretchable sensor components having a plurality of electrodes placed in predetermined locations. The forehead patch 500 includes at least three electrodes configured for EEG sensing, shown as first EEG recording electrode 502a and second EEG recording electrode 502b along with a common ground electrode 506 and a common reference electrode 508. The forehead patch 500 further includes at least three electrodes configured for EOG sensing, shown as first EOG recording electrode first EOG recording electrode 504a and second EOG recording electrode 504b along with the common ground electrode 506 and the common reference electrode 508.

The chin patch 520 includes an EMG recording electrode 522, a common ground electrode 524, and a common reference electrode 526. In some implementations, the device 500 (e.g., the forehead patch 500 or the chin patch 520) includes only four electrodes including two recording electrodes (e.g., two EEG recording electrodes, two EMG recording electrodes, two EOG recording electrodes, one EEG and one EMG, one EEG and one EOG, or one EOG and one EMG recording electrode), one common ground electrode, and one common reference electrode. In some implementations, the device 500 (e.g., the forehead patch 500 or the chin patch 520) includes only three electrodes include one recording electrode (e.g., an EEG, EOG, or EMG recording electrode, a common ground electrode, and a common reference electrode).

The targeted locations of electrodes 502, 504, 506, 508, 522, 524, and 526 were chosen for measuring EEG, EOG, and chin EMG, by following the PSG setup and the standards from AASM [31].

Each of the forehead patch 500 and the chin patch 520 further include at least one circuit. For example, a first circuit 510 is coupled to the forehead patch 500 and a second circuit 528 is coupled to the chin patch 520.

FIG. 5B shows a diagram of the circuitry, operation, and analysis of the exemplary devices 500 and patches 500, 520. Both circuits 510, 528 of the forehead patch 500 or the chin patch 520 are shown in the flowchart of FIG. 5B as circuit 530. The circuit 530 includes a multi-channel differential amplifier 532 in electrical communication with each of the electrodes of the forehead patch 500 and/or the chin patch 520. The circuit 530 further includes a Bluetooth-low-energy microcontroller 534 in communication with the amplifier 532. Examples of the amplifier 532 are shown for the forehead and the chin patches using an integrated circuit. The IC (ADS1299 and ADS 1292) includes a multi-channel differential amplifier with 24-bit ADC. The amplifier IC 532 interfaces to a Bluetooth-integrated microcontroller. The circuit 530 further includes an antenna 536 (e.g., a 2.4 GHz antenna) configured to communicate with a remote device 540 (e.g., mobile device 460 of FIG. 4; a smart phone or tablet or other device configured for wireless data transmission and storage). In some implementations, the remote device is any other personal computing device having a user interface configured to display at least one of the transmitted data and the sleep score and configured with a short-range communication interface. The circuit 530 also includes a rechargeable Li-polymer battery 538 providing power to the circuit 530.

The remote device 540 includes (or transmits data to) an analysis system 550 (e.g., similar to analysis system 462 of FIG. 4). The analysis system 550 (e.g., a remote analysis system or wireless monitoring system) is separate from the wearable biopatch device 500. The analysis system 550 includes a data processing and segmentation step 552 configured to analyze the signals in real time and a convolution neural network (CNN) 554. The CNN 554 is configured to classify a sleep stage or detect a sleep apnea event based on the incoming EEG, EOG, and/or EMG signals from the device 500 (e.g., the forehead patch 500 or chin patch 520). The remote analysis system 550 is configured to receive signals from the wearable biopatch device 500 and either (i) relay the received signals to a cloud analysis system having the trained machine learning or neural network or (ii) perform the analysis with a locally executing trained machine learning or neural network.

In some implementations, the remote analysis system or the cloud analysis system is configured to provide a sleep score associated with the sleep stage classification and apnea event detection. In some implementations, a system is disclosed comprising the wearable biopatch device of the previous examples and an analysis system of the previous examples. In some implementations, the analysis system comprises cloud infrastructure.

### Example Method of Operation

Disclosed herein is a method comprising: (i) providing a wearable biopatch device as described in the above examples, the biopatch including: a hypoallergenic silicone adhesive substrate comprising an inner surface and an outer surface; an integrated sensor system coupled to the inner surface of the silicone adhesive substrate, the integrated sensor system comprising a plurality of electrodes placed in predetermined locations and electrical connections between the plurality of electrodes, including at least three electrodes configured for EEG sensing and at least three electrodes for EOG sensing; a silicone fabric substrate disposed on the outer surface of the silicone adhesive substrate, the hypoallergenic silicone adhesive substrate and silicone fabric substrate providing conformal and stretchable substrate for conformal and stretchable contact on a facial area; and at least one circuit configured for wireless data transmission disposed on a portion of the silicone fabric substrate, the at least one circuit comprising thin, flexible film connectors; (ii) sensing, via the plurality of electrodes, EEG signals from the at least three electrodes configured for EEG sensing and EOG signals from the at least three electrodes configured for EOG sensing over a period spanning at least one REM cycle; (iii) transmitting the sensed EEG signals and the sensed EOG signals to local computing device; (iv) processing and segmenting, at the local computing device, the sensed EEG signals and the sensed EOG signals; (v) analyzing, via the local computing device or a remote analysis system that received the data from the local computing device, the segmented EOG signals and the segment EEG signals via a trained machine learning operation (e.g., convolutional neural networks (CNN)); and (vi) providing a sleep score associated with a sleep disorder to a graphical user interface (e.g., associated with the local computing device) based on the analyzed brain activity data.

In some implementations, the method further includes: (vii) classifying, based on the sleep score, a sleep stage; and (viii) detecting, based on the sleep score, a sleep apnea event.

In some implementations, the at least three electrodes associated with the EEG sensing are placed on the forehead to acquire the EEG signals, and the at least three electrodes associated with the EOG sensing are placed at the temple to acquire the EOG signals.

In some implementations, the wearable biopatch device is self-applying to be installed by the subject, and wherein the EEG signals and EOG signals are monitored by a user at a remote location.

### Experimental Results and Additional Examples

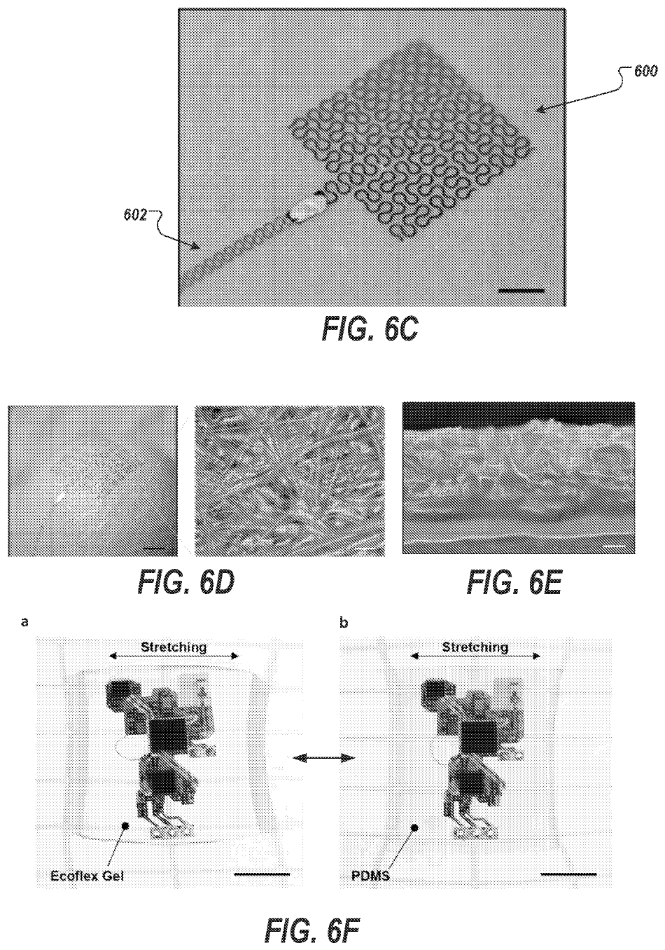

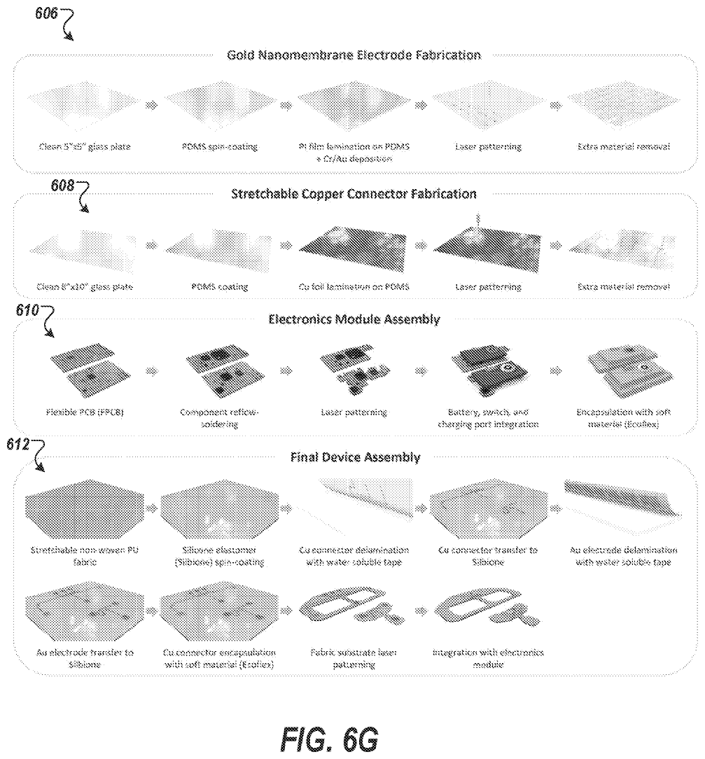

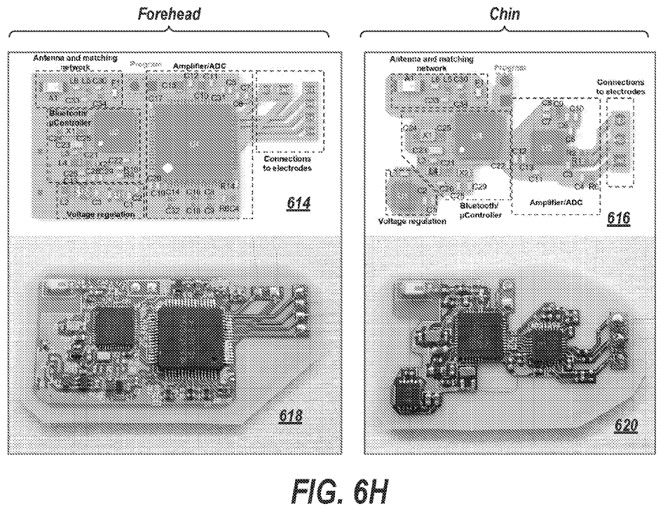

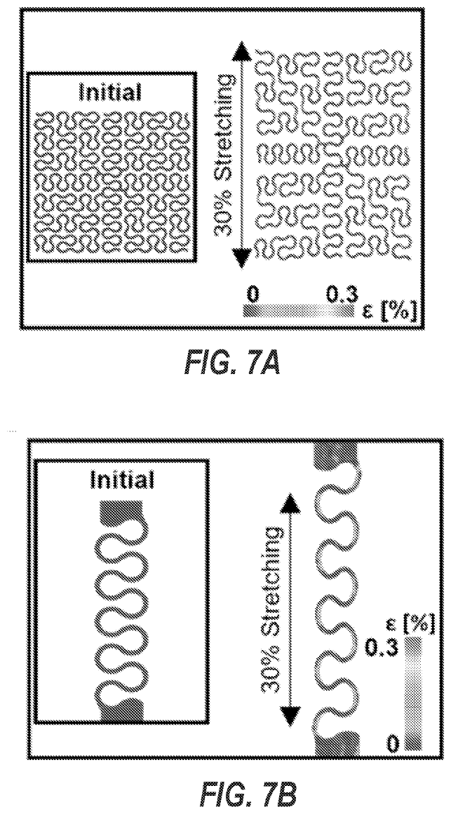

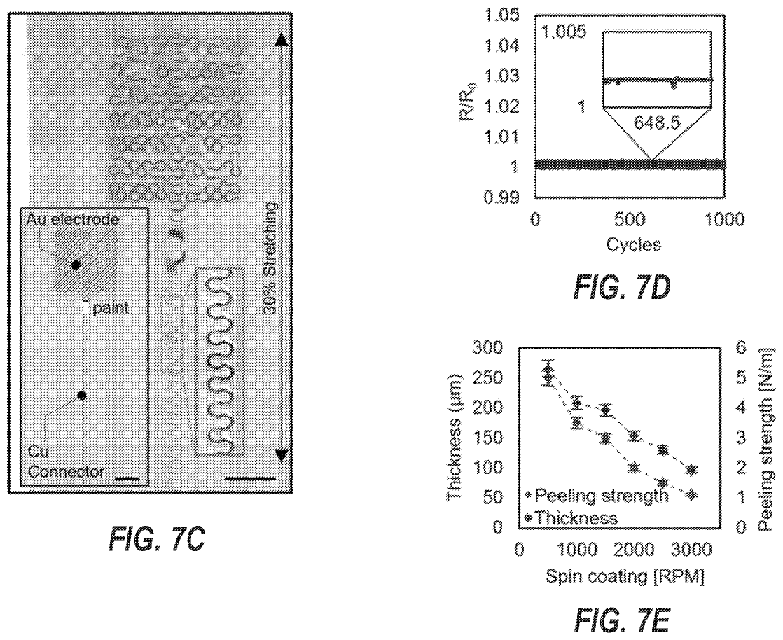

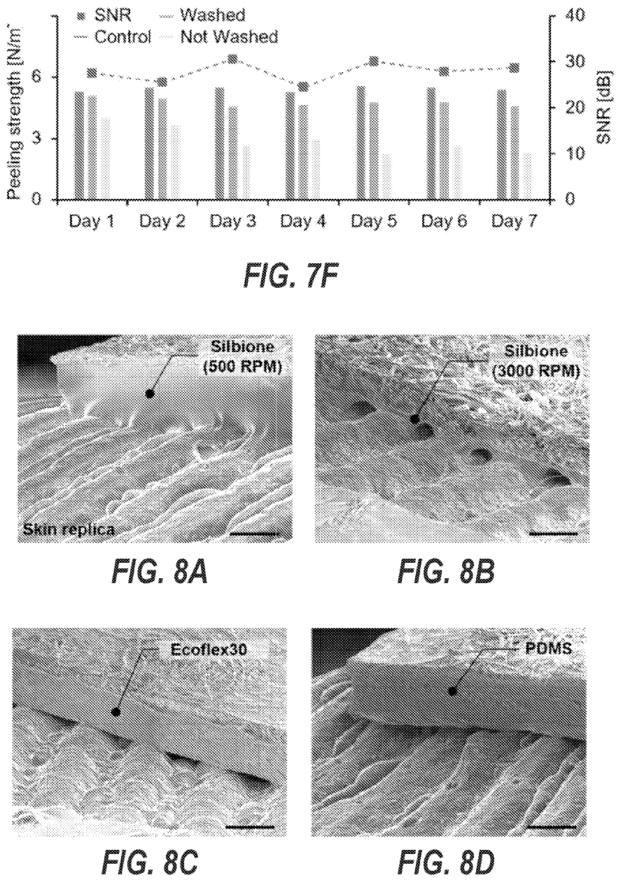

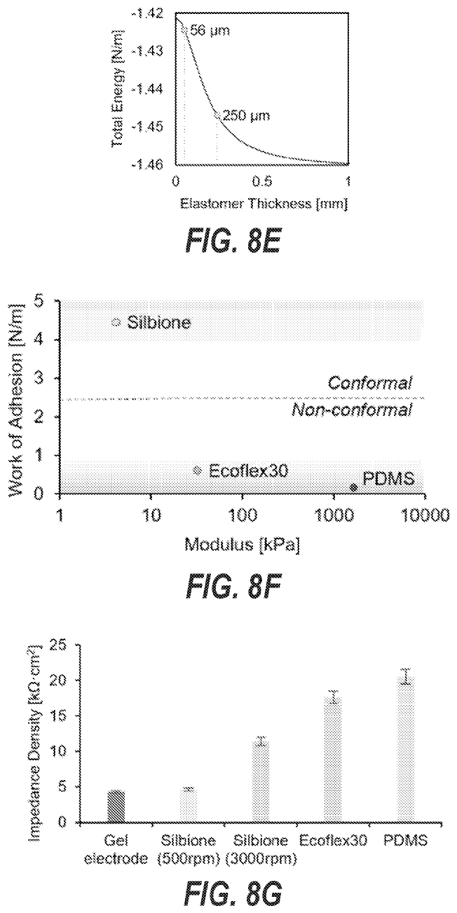

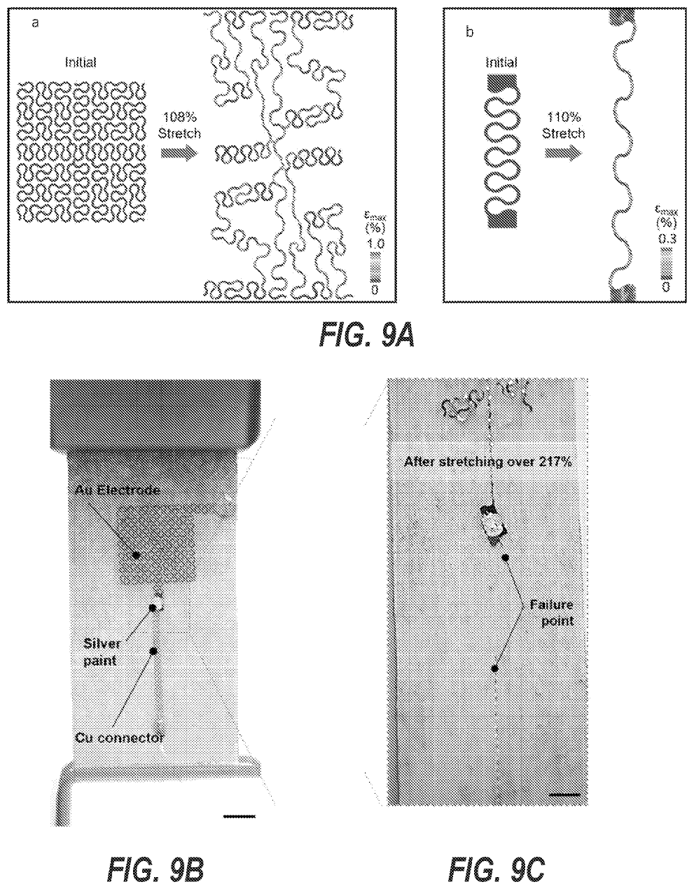

A study was conducted to develop a wearable biomedical system that offers at-home wireless sleep monitoring for the clinical assessment of sleep quality and sleep apnea, e.g., as described in relation to FIGS. 1-4. FIGS. 6-16 show the aspects various of the prototyped device and analysis system.

The study is understood to be the first demonstration of automated detection of sleep disorders using a multi-sensor integrated patch and deep learning algorithm. The prototyped wearable platform showed the potential for convenient, reliable, and accurate sleep monitoring and analysis with enhanced accessibility and effectiveness in which the system provides a multimodal physiological measurement with seamless and unobtrusive integration with skin. The study employed a new combination of materials and fabrication processes to provide reliable integration and usability of the system (for potentially home self-administration) while offering scalable manufacturing of a large-area system. The study also performed a pilot clinical study that compared the standard PSG to the prototyped system that highlight the high signal quality and feasibility of both visual and automated sleep scoring of the system. Training on existing datasets demonstrated the device's potential to detect obstructive sleep apnea-related arousals. CNN-based data analysis methods for automated sleep scoring and apnea detection could further validate the example system's applicability with high performance.

### Example Stretchable Electrodes and Interconnects of the Wearable Forehead and Chin Sensor

FIG. 6A shows images of a fabricated array of stretchable gold electrodes for the exemplary wearable biopatch device. FIG. 6B shows an image of fabricated copper stretchable interconnectors and a corresponding microscopic image of the interconnectors, according to one implementation. FIG. 6C shows an image of an example electrode with interconnector on a fabric surface. FIG. 6D shows an image of an example electrode with interconnector disposed on a curved surface along with an optical microscope image of the fabric fibers.

Serpentine Electrodes—As shown in FIGS. 6A-6D, at least a portion of the electrodes 600 (e.g., electrodes 150 of FIG. 1; electrodes 502, 504, 506, 508, 522, 524, and 526 of FIGS. 5A-5B) was fabricated to be stretchable. All of the plurality of electrodes 600 in the device 100 were stretchable and formed in a meandering or serpentine structure. Experimental results show the serpentine electrodes 600 offer improved formability and stretchability for molding and forming to the contours of a facial area of a user.

Example Fabrication of Serpentine Electrodes—The electrodes 600 were fabricated as gold membrane electrodes formed by laser micromachining. It was contemplated that graphene electrodes or other material could be used. A femtosecond laser process was employed that offered high-precision processing of various materials and high-throughput manufacturing of complex structures [23].

The photos in FIG. 6A show a fabricated array of gold electrodes on a 5-inch square plate and a corresponding microscopic image of the electrode 600 with curved patterns, providing enhanced stretchability and mechanical reliability [23, 24, 32]. The laser spot size used in the fabrication was 13 μm, and the pattern width of each electrode was 124 μm (to provide sufficient area for skin contact).

The photos in FIG. 6B show a set of fabricated copper interconnectors 602 (e.g., as in FIG. 1, the integrated sensor system 120 is coupled to the inner surface 112 of the silicone adhesive substrate 110) on a large glass (8-by-10-inch), which makes an electrical connection between multiple electrodes and integrated circuits. Copper connectors can offer enhance solderability and robust electrical connections with the device, as shown in the closeup image in FIG. 6B. The connector's pattern width was 79 μm, and the laser spot size was 18 μm. The laser spot size was larger than the electrode case of FIG. 6A because higher laser power and repetition were required due to copper's higher thermal conductivity and thickness. Overall, the laser micromachining of electrodes and interconnectors enables fast, reliable, and scalable manufacturing capabilities.

The electrodes 600 were connected with the stretchable wire via soldering to maintain mechanical stability when mounted on a soft fabric as shown in FIG. 6C. In contrast to the conventional woven fabric, the fabric composite provides omnidirectional elasticity as shown in FIG. 6D when the electrode 600 is disposed on a curved surface (e.g., a 1-inch diameter sphere). The fabric composite provides convenient processibility without untangling due to its non-woven structure composed of a random network of fibers, as shown in the closeup, optical microscope image of FIG. 6D.

The soft silicone adhesive (Silbione) can additionally penetrate through the networks of fabric fibers to provide mechanical interlocking for robust integration of the bilayer while leaving the top side of the fabric dry and non-sticky for handling, as shown in the cross-sectional optical microscope image of the fiber in FIG. 6E.

As shown in FIGS. 6F, the soft packaging method using the fabric can provide strain isolation to avoid mechanical damage to the electronics, e.g., during device assembly, handling, and multiple uses during sleep [33-35]. FIG. 6F (having subpanels A and B) shows images of stretching the circuit with and without the strain isolation layer. The low modulus Ecoflex Gel layer reduces the stress of the circuit by releasing its stress to proper strain.

### Example Fabrication of an Example Wearable Patch

FIG. 6G provides details and illustrations of fabrication and assembly of an example sleep patch, including gold nanomembrane mesh electrode fabrication, stretchable solderable copper connector fabrication, electronics module integration assembly and packaging with soft material, and fabrication of the fabric substrate and the final device assembly.

Fabric substrate fabrication. To form the silicone elastomer, e.g., in process 612, Parts A and B of Silbione (A-4717, Factor II Inc.) were mixed with 1:1 weight ratio for 5 minutes. To make a uniform thickness of the adhesion layer, mixed uncured Silbione was poured on polytetrafluoroethylene (PTFE) sheet and spin-coated at 500 RPM for 1 minute. Brown fabric medical tape (9907T, 3M) was placed on the uncured Silbione surface and followed by curing process in an oven at 65° C. for 30 minutes. After curing of Silbione, PTFE sheet was detached.

Nanomembrane electrode fabrication. To fabricate the gold electrode array, e.g., in process 606, gold electrodes were fabricated by E-beam evaporation and facile laser cutting. PDMS (Sylgard 184, Dow) was utilized for the bottom layer of electrode fabrication since it offers both proper adhesion and easy release feature for this process. A polymer film (18-0.3F, CS Hyde) was laminated on the cured PDMS surface. An electron-beam deposition process was used to deposit gold on the film. A precise laser-cutting process was applied to the film to get a stretchable serpentine pattern of the electrode. Finally, non-functional materials besides the electrode patterns were removed by delaminating from PDMS surface.

Stretchable connector fabrication. To fabricate the stretchable interconnect, e.g., in process 608, a thin copper foil with laser cutting process enabled a scalable electrical connector fabrication. PDMS was coated and cured on a glass plate (8″×10″). 6 μm-thick copper foil (BR0214, MSE Supplies LLC) was laminated on the cured PDMS surface. A laser-cutting process was applied to the copper foil to get a stretchable serpentine pattern of the electrical connector. Then, the rest of the Cu foil beside the pattern was removed and delaminated from the PDMS surface.

Fabrication of circuits and encapsulation. To fabricate the circuit components, e.g., in process 610, the circuits used a flexible PCB board (FPCB). All electronic components were mounted on the board with a reflow solder process (see FIG. 6H). To enhance the mechanical flexibility of the circuit, unnecessary areas were removed with laser cutting. For power supply and management, a lithium polymer battery assembly was used with a slide switch and a circular magnetic recharging port. A low-modulus elastomer (Ecoflex GEL, Smooth-On) was placed underneath the integrated circuit as a strain-isolation layer. The overall electronic system was encapsulated and soft-packaged with an additional elastomer (Ecoflex 00-30, Smooth-On), leaving only the switch and charging port exposed.

Device assembly. In process 612, both the copper connector and gold electrode were transferred to the soft adhesive side of the fabric with water-soluble tape (ASW-35/R-9, Aquasol corporation). Silver paint was applied between the copper connector pad and gold electrode pad. Silver paint offers robust mechanical/electrical connections beyond the yield point of the electrode system. The applied silver paint was dried in an oven at 65° C. for 30 minutes. The copper connector and the silver paint were encapsulated with Ecoflex, which was cured in an oven at 65° C. for 180 minutes. Electrode system mounted fabric was patterned by laser cutting process. The soft-packaged electronic system was attached on the fabric side of the fabric substrate by adding and curing a thin silicone layer.

Prototyped Circuit Design—FIG. 6H shows an example implementation of the control and communication circuit (e.g., circuit 140 of FIG. 1) for the forehead (614) and chin (616) patches. The top view shows the circuit component along with annotations of the functional blocks, while the bottom photo shows an example of the fabricated circuit of the fully assembled circuit of the system's forehead (618) and chin (620) patch.

### Example Device Performance and Characterization

Computational modeling. The study characterized the mechanical deformations and strain distributions associated with the system on human skin using three-dimensional FEA. The study used eight-node 3D solid elements to simulate the fabric and Silbione and adopted four-node shell elements with a two-layer (gold/PI) for electrode and copper/PI for connector), with optimized meshes to assure computational precision. The study assigned displacement-type boundary conditions to the fabric's side surfaces where varying amounts of stretching were applied. Elastic stretchability was defined as the point at which the maximum strain in the metal layer exceeds the yield strain (0.30% for copper and 1% for gold) throughout at least half of the width of any segment. The Young's modulus (E) and Poisson's ratio (v) of the materials used in the simulations included Eₐᵤ=78 GPa and vₐᵤ=0.44 for gold, Eₚᵢ=2.5 GPa and vₚᵢ=0.34 for polyimide, E_{cu}=119 GPa and v_{cu}=0.34 for copper, under fabric (E_{f}=1.28 MPa, v_{f}=0.184) with Silbione (Eₛᵢ=5 kPa, vₛᵢ=0.48).

Experimental study of mechanical reliability. The study performed mechanical characterization of fabric's elasticity. The study mounted fabric with 30×80 mm on a motorized testing machine (ESM303, M5-5, Mark-10) and gently stretched with 50 mm/min up to its failure point. The tests were conducted in 3 directions of fabric (Roll-direction, 45-degree, and vertical direction). Young's modulus and Poisson's ratio were calculated using the average data of three directions before 100% stretching. For the cyclic test and failure test of electrode system, the study used an electrode system that consisted of a gold electrode and copper connector prepared on a fabric substrate. The study applied silver paint to make a mechanical/electrical connection between the gold electrode and the copper connector. 100 μm-diameter copper wires were attached to both edge pads of the Cu connector and the designed pad of the gold electrode to measure their electrical resistance change. Electrical resistance was measured by a LCR meter (Model 891, BK Precision). For the cyclic stretching test, the electrode system was repeatedly stretched and relaxed with 150 mm/min speed for 1,000 cycles. The failure test was conducted with 50 mm/min speed stretching on the system up to its electrical failure.

Imaging-based study of conformal contact. For Silbione-fabric substrate thickness measurement, the study measured and compared the thickness of the Silbione-fabric substrate depending on coating condition. Silbione was spin-coated with various coating speeds (500, 1000, 1500, 2000, 2500, and 3000 RPM for 1 minute). Each sample was cut into 5×10 mm size by razor blade to measure its cross-section. Optical image analysis was performed using 3D Surface Profiler (VK-X3000, Keyence).

For conformal contact visualization and comparison, the study compared the level of conformal contact visually. A Thermo Axia Variable Pressure SEM was used to take images of the microstructure of the skin-attached adhesion layer. As the compared adhesion layers, thick Silbione (500 RPM spin-coated), thin Silbione (3000 RPM spin-coated), Ecoflex 00-30, and PDMS was cured on the fabric substrate and placed on human skin replica which is fabricated by a casting process (EpoxAcast™ 670 HT, Smooth-On).

Study of a device's peeling strength and reusability. For peeling strength measurement and calculation, the study used test adhesive pads of various thicknesses (56, 73, 99, 146, 172, and 250 μm) that were prepared in 30×80 mm size. For the peeling strength analysis of the fabric substrate, the fabric substrate was attached to the skin and peeled in the vertical direction with a motorized force tester (ESM303, M5-5, Mark-10). The motorized force tester recorded adhesion force data during the test. The adhesive pad was peeled mechanically from the skin at a speed of 50 mm/min. The average peel strength (N/mm) was calculated by measuring the average load (N) of the peel test and dividing it by the width (mm) of the bonded pad. Each test was repeated three times to evaluate the average peeling strength.

Experimental setup of reusability and method of soap washing. The study evaluated the reusability of the Silbione adhesive pad with the 250 μm-thickness by proper cleansing protocols. The test samples were attached on the forehead for seven hours during sleep and were tested repeatedly for seven days. For preparing the washed sample, 50 ml warm water (35° C.) was mixed with 5 ml dish detergent, and the swab was dipped into the solution to soak the prepared detergent solution. To remove residue from the adhesive after each attachment to the forehead, the washed sample was gently brushed with a wet swab for 1 min. Two additional group experiments (Unwashed pad after each attach/detach cycle, newly fabricated adhesion pad) were conducted to evaluate the efficacy of the cleansing method. Each sample's peeling strength for each day was measured by motorized force tester (ESM303, M5-5, Mark-10). Samples were peeled from the skin at 50 mm/min speed. The peel strength (N/mm) was calculated by measuring the load (N) of the peel test and dividing it by the width (mm) of the bonded pad.

Measurement of work of adhesion. The test consisted of indenting an adhesive layer (Silbione, Ecoflex 00-30, and PDMS) mounted circular polished steel probe into a pig skin that was firmly bonded to a larger circular steel substrate. The probe with adhesive layer was brought into contact with the skin for a minute and the probe was then retracted at a given speed. The diameter of the probe was 20 mm. The speed of the probe was 0.4-40 mm/min. The normal direction peeling force and displacement of the probe were measured by a motorized force tester (stage: ESM303 and gauge: M5-5, MARK-10). The stress-strain curves were plotted from the measured data and debonding energy of each condition was calculated through it. The work of adhesion of each material was derived by linear fitting of the plot.

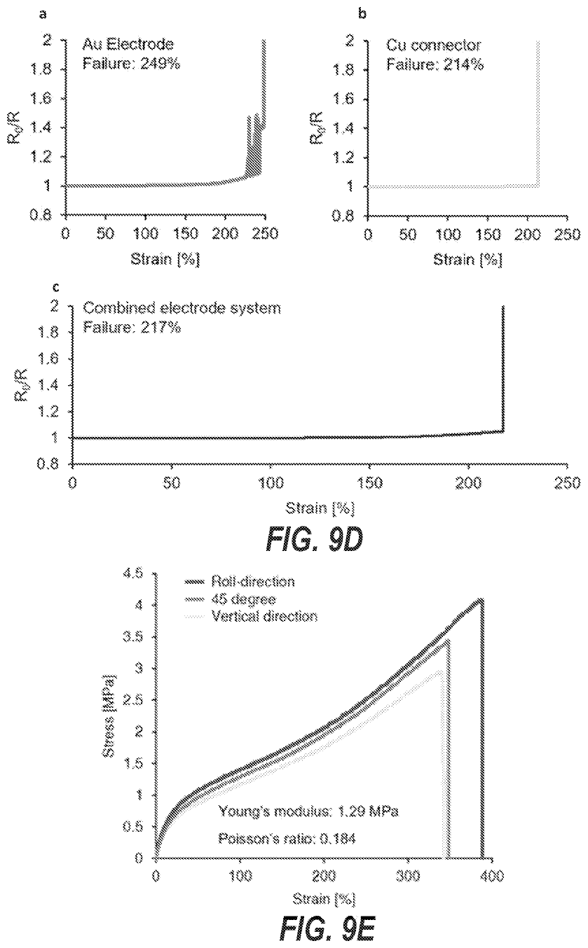

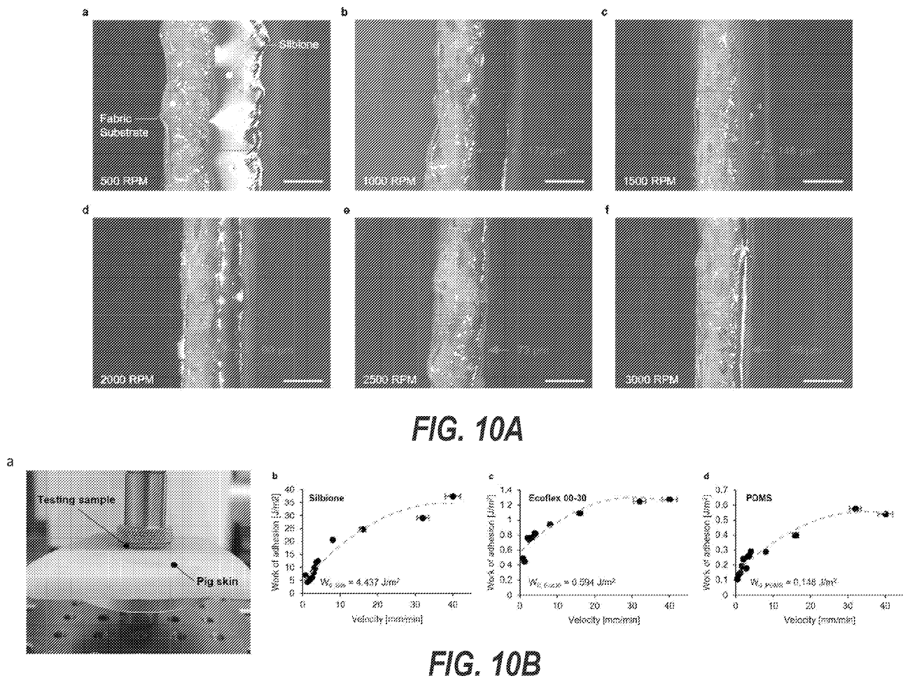

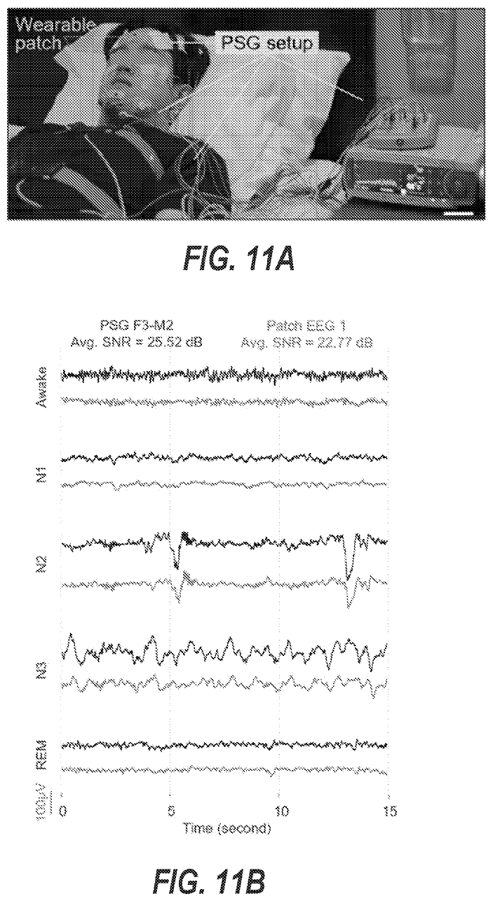

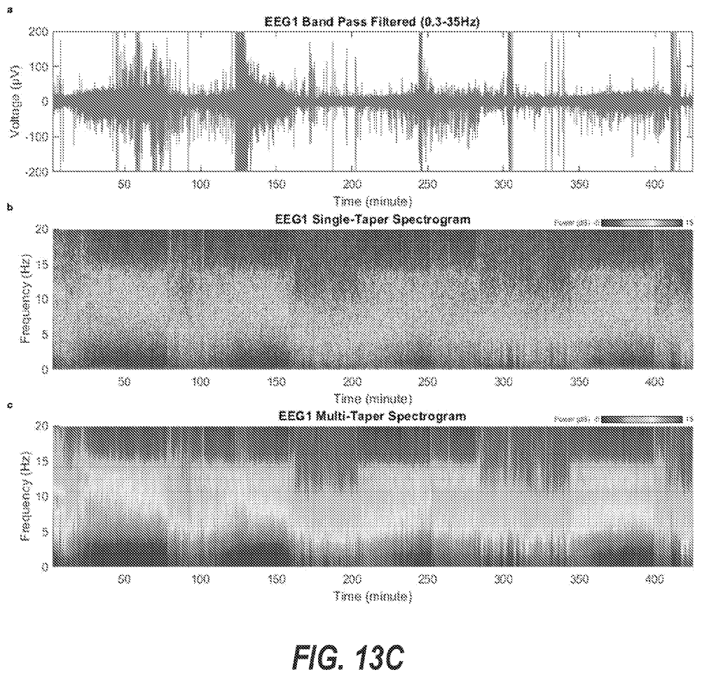

Analysis of skin-electrode contact impedance and SNR. To compare the skin impedance of the electrode with different adhesion layers, gold electrode was placed on various substrates (500 RPM spin-coated Silbione, 3000 RPM spin-coated Silbione, Ecoflex 00-30, PDMS). Also, the control group experiment (gel electrode) was conducted to compare electrode-skin contact impedance. The test was conducted on the forearm area and the electrode attached area was properly cleaned with skin preparation gel (NuPrep Skin Prep Gel, Weaver & Co.). Electrode-skin contact impedance was measured by a skin impedance meter (Model 1089NP Checktrode, UFI) which was connected to two electrodes on skin. Normalized value could be achieved by calculating the impedance density of each sample. The effective area of the serpentine gold electrode and gel electrode were measured by 3D Surface Profiler (VK-X3000, Keyence). For the SNR calculation of the sleep data, the noise was assumed to be the data measured before eye closure without any activity, and its amplitude was calculated. For SNR calculation for reusability analysis in FIG. 7F, amplitudes of 100-second-long delta wave during the first N3 stages were calculated from each measurement. The signal amplitudes were averaged for each day of the two separate 7-day measurement. For SNR comparison in FIG. 11B, amplitudes of shown signals at each sleep stage were calculated and averaged. The SNR value was calculated with following equation:

$$S N {R}_{d B} = [ {( \frac{{A}_{signal}}{{A}_{noise}} )}^{2} ] .$$

Human subject study. Once the standard PSG setup was placed on the patient by a sleep study technician, the devices were placed on the patient's face in a way as to not disrupt or overlap with the PSG settings. The sampling rate of PSG was 200 Hz, and the sampling rate of the study's system was 250 Hz. Data from these patients were used to compare sleep-wake staging between gold-standard PSG and the example device, using both traditional visual scoring and automated analyses. Instruction was given to the subjects regarding how to use the devices by themselves, and the devices were delivered to the subjects to take sleep measurement at their home.

Manual scoring of sleep stages and comparison with PSG—A sleep technologist manually scored the data from the eight clinical study patients measured with both PSG and the example system according to the AASM guideline. The patient information of the data was blinded to the sleep technologist. Malfunction of the device occurred during the measurement from four of the patients, and the epochs measured during the malfunction were excluded from the analysis. 2,228 epochs were excluded, and 4,970 epochs were used for the comparison analysis. The scoring results of the 4,970 epochs measured with both PSG and the example system from the eight patients were used to calculate the agreement and Cohen's kappa coefficient.

Data processing—All data processing was done with MATLAB. The data measured with the system were first processed by bandpass filter and notch filter. The cut-off frequencies of the bandpass filter recommended by the AASM guideline was used: 0.3-35 Hz for EEG and EOG, and 10-100 Hz for EMG. Stop band frequencies of the notch filter was set to 59-61 Hz to remove the power line noise with 60 Hz. The filtered data was used to generate multi-taper spectrogram. The recommended parameters from Prerau et al. were adopted. The frequency range of the spectrogram was set to 0-20 Hz. The time-half-bandwidth product was set to 5, and the number of tapers was 9. Window size was set to 5 seconds, and the step size was 1 second.

Classification of sleep stages and apnea—The details of machine learning layer information for sleep stage classification appear in below in Tables 1 and 2.

TABLE 1
Machine learning layer information for sleep stage classification.
Detailed information about the layers of proposed network
for sleep stage classification.

| | | # | Kernel | |
|---|---|---|---|---|
| Layer | Output | filter | size | Option |
| Input | 256 × 256 × 3 | | | |
| Resize | 64 × 64 × 3 | | | |
| Conv2D-1 | 64 × 64 × 112 | 112 | 3 × 3 | Stride: 2 × 2 |
| | | | | Mode = Same |
| BatchNorm | 64 × 64 × 112 | | | |
| Leaky_Relu | 64 × 64 × 112 | | | |
| MaxPool2D-1 | 32 × 32 × 112 | | 2 × 2 | Stride: 2 × 2; |
| | | | | Mode = Valid |
| Conv2D-2 | 32 × 32 × 32 | 32 | 3 × 3 | Stride: 2 × 2 |
| | | | | Mode = Same |
| BatchNorm | 32 × 32 × 32 | | | |
| Leaky_Relu | 32 × 32 × 32 | | | |
| MaxPool2D-2 | 16 × 16 × 32 | | 2 × 2 | Stride: 2 × 2; |
| | | | | Mode = Valid |
| Conv2D-3 | 16 × 16 × 48 | 48 | 5 × 5 | Stride: 2 × 2 |
| | | | | Mode = Same |
| BatchNorm | 16 × 16 × 48 | | | |
| Leaky_Relu | 16 × 16 × 48 | | | |
| MaxPool2D-2 | 8 × 8 × 48 | | 2 × 2 | Stride: 2 × 2; |
| | | | | Mode = Valid |
| Dropout | 8 × 8 × 48 | | | ρ = 0.45 |
| Flatten | 3072 × 1 | | | |
| FullyConnected | 512 × 1 | | | |
| BatchNorm/Dropout | 512 × 1 | | | ρ = 0.45 |
| FullyConnected | 64 × 1 | | | |
| BatchNorm/Dropout | 64 × 1 | | | ρ = 0.45 |
| Softmax | 5 × 1 | | | |
| Output | 5 × 1 | | | |

TABLE 2
Machine learning layer information for apnea detection.
Detailed information about the layers of proposed
network for sleep apnea detection.

| Layer | Output | # filter | Kernel size | Option |
|---|---|---|---|---|
| Input | 224 × 224 × 3 | | | |
| Conv2D-1 | 213 × 213 × 64 | 64 | 12 × 12 | Stride: 1 × 1 |
| BatchNorm | 213 × 213 × 64 | | | |
| Relu | 213 × 213 × 64 | | | |
| MaxPool2D-1 | 27 × 27 × 64 | | 8 × 8 | Stride: 8 × 8; |
| | | | | Mode = Same |
| Dropout | 27 × 27 × 64 | | | ρ = 0.5 |
| Conv2D-1 | 16 × 16 × 64 | 64 | 12 × 12 | Stride: 1 × 1 |
| BatchNorm | 16 × 16 × 64 | | | |
| Relu | 16 × 16 × 64 | | | |
| MaxPool2D-1 | 4 × 4 × 64 | | 4 × 4 | Stride: 4 × 4; |
| | | | | Mode = Same |
| Dropout | 4 × 4 × 64 | | | ρ = 0.5 |
| Flatten | 1024 × 1 | | | |
| GRU | 9 × 1 | | | Hidden: 9 |
| Dropout | 9 × 1 | | | ρ = 0.5 |
| FullyConnected | 2 × 1 | | | |

Segmented images of the multi-taper spectrogram were used for training and evaluation of the CNN-based sleep stage classification and apnea event detection. Sleep data from 32 healthy subjects with 15,590 epochs were used to train the CNN-based sleep stage classification. Out of the 15,590 epochs, 1883 were W, 685 were N1, 5858 were N2, 4404 were N3, and 2760 were R. Sleep data from 40 apnea patients with 35,927 epochs obtained from ISRUC public PSG dataset were used to train the CNN-based apnea event detection. Out of 35,927 epochs, 829 epochs contained apnea events, and 35,098 epochs were labeled as normal. Sleep data from 8 patients with 4,970 epochs from the clinical study were used as the test dataset to evaluate the performance of the CNN-based classification models. The labels of sleep stage and apnea events were made on each epoch. For sleep stage label, out of the 4,970 epochs, 1,141 were W, 343 were N1, 2,423 were N2, 495 were N3, and 568 were R.

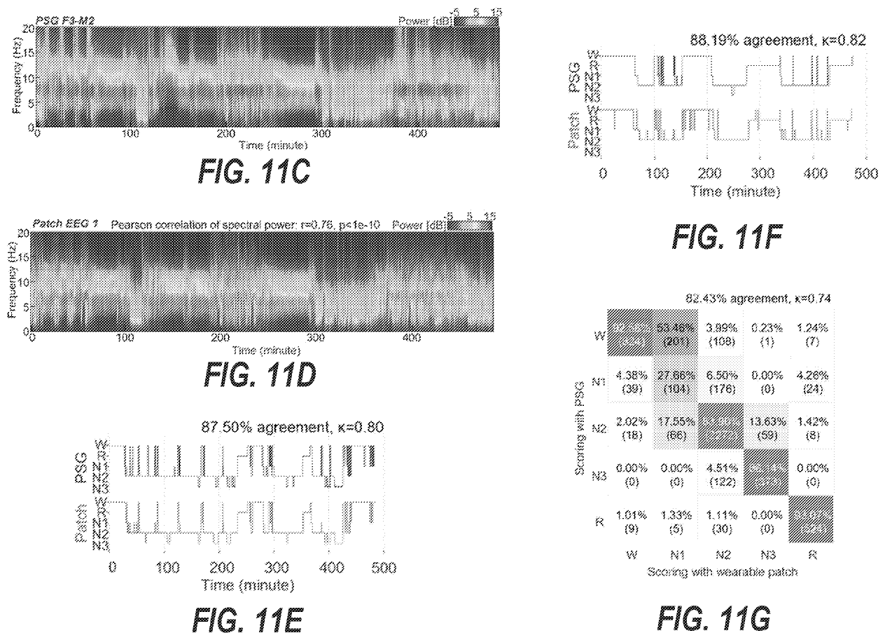

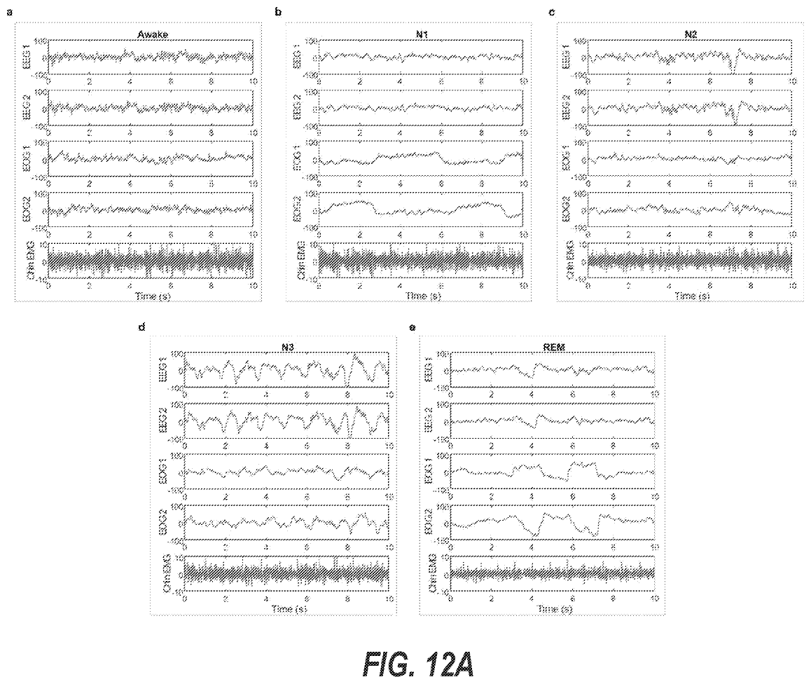

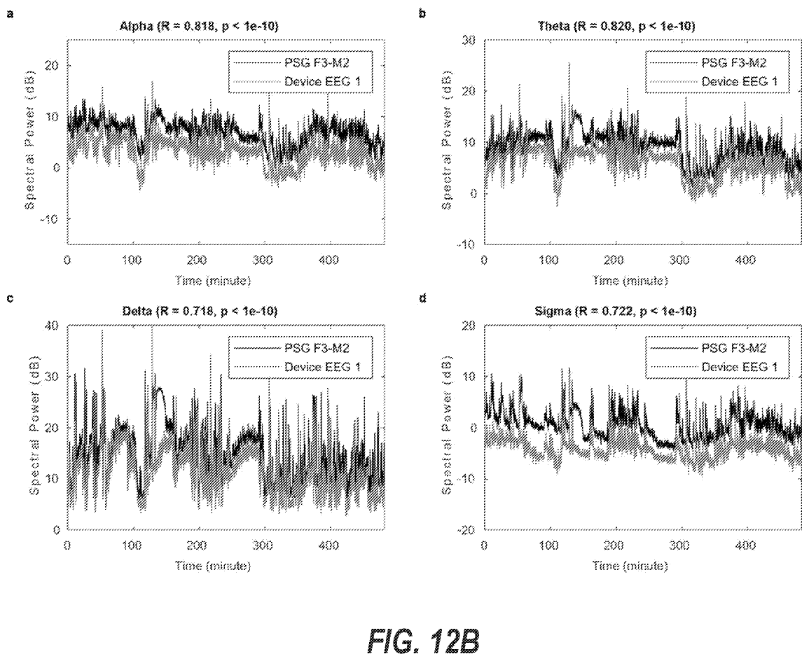

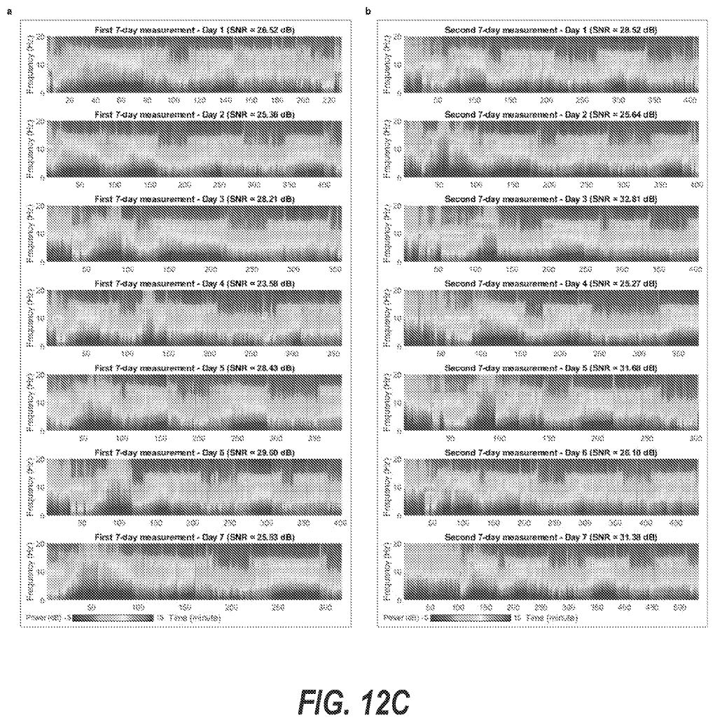

For apnea event label, out of the 4,970 epochs, 196 epochs contained apnea events, and 4774 epochs were labeled as normal. CNN architectures were created according to the form of input data, image-based multi-taper spectrograms, drawing influence from earlier models [43]. The inputs of the example CNN were epoch-by-epoch 30-second-long spectrogram images (128×128 pixels) of 4 channels (two EEG and two EOG channels) combined in a square image (256×256 pixels). The spectrogram image was then resized into 64×64 and converted to values between 0 and 1 using normalization. Since the color-image consists of 3 color layers (red, green, and blue), every input matrix dimension was a 3-dimensional matrix (64×64×3). The non-linear activation functions employed are the Leaky Rectified Linear Unit (Leaky ReLU). ADAM (learning rate=0.002) was utilized for the optimization of the CNN architecture, and the error was calculated using the cross-entropy loss function. The batch size was set to 16 and the dropout deactivation rate was set to 0.5. Early stopping was used to prevent overfitting by randomly eliminating 20% of the data from the training set and utilizing it as a validation set at the start of the optimization phase. When the validation loss stopped improving, learning rate annealing was performed with a factor of 5. The training was terminated when two successive decays occurred with no network performance improvement on the validation set and hyper-parameters were optimized by random selection method. Resized data was entered into CNN, featuring three layers of two-dimensional convolutions with filters of size 112, 32, and 48, respectively and kernel size 3×3, 3×3, and 5×5, respectively. A single convolutional cell (Conv_N) consisted of a convolutional layer, one layer of Batch Normalization, one layer of Max pooling step with a filter size of 2, and one layer of Leaky ReLu function. Lastly, the data was flattened and followed by two fully connected layers and passed through a softmax layer and finally outputted the predicted class (one of the five sleep stages). Details of the structure and parameters of the CNN for sleep stage classification are further summarized in FIG. 8, illustrating example machine learning architecture for sleep stage classification. Table 3 below also displays details on sleep state classification performance and comparison of recent EEG-based wearable sleep monitoring devices.

TABLE 3
Sleep stage classification performance comparison of
recent EEG-based wearable sleep monitoring devices.

| | # of | # of | Automated | Classification accuracy of each | | | | | Overall |
|---|---|---|---|---|---|---|---|---|---|
| | measured | subjects/ | scoring | sleep stage (%) | | | | | accuracy |
| Ref. | signals | classes | algorithm | W | N1 | N2 | N3 | R | (%/kappa) |
| This | 2 EEG, 2 | 40/5 | CNN | 88.2 | 52.3 | 84.8 | 86.4 | 76.9 | 83.89/0.76 |
| work | EOG, 1 | | | | | | | | |
| | EMG | | | | | | | | |
| [1] | 2 EEG, | 1/4 | Decision | — | — | — | — | — | 74/— |
| | EOG, 1 | | tree | | | | | | |
| | EMG | | | | | | | | |
| [2] | 4 EEG | 10/5 | SVM | 84.1 | 22.4 | 85.0 | 87.1 | 83.2 | 76.7/0.69 |
| [3] | 2 EEG, 1 | 47/5 | Rules and | 80.9 | 22.9 | 79.7 | 74.9 | 71.5 | 71.3/0.63 |
| | EOG | | thresholds | | | | | | |
| [4] | 7 EEG | 661/5 | LSTM | 74.0 | 47.7 | 82.9 | 82.6 | 84.5 | 83.5/0.75 |
| [5] | 2 EEG | 16/5 | SVM | 74.3 | 4.9 | 84.1 | 74.7 | 45.8 | 74.1/0.61 |
| [6] | 8 EEG | 20/5 | Random | 83.8 | 52.0 | 78.8 | 92.5 | 76.7 | 80.5/0.73 |
| | | | forest | | | | | | |
| [7] | 18 EEG | 15/5 | Random | — | — | — | — | — | 70/0.58 |
| | | | forest | | | | | | |

For apnea event, an architecture combining CNN and Gated Recurrent Unit (GRU) was created. The inputs of the architecture were epoch-by-epoch 60-second-long spectrogram images (128×128 pixels) of 4 channels (two EEG and two EOG) combined in a square image (256×256 pixels). For the 60 seconds of data included in each spectrogram, 30 seconds of corresponding epoch, and the next 30 seconds of the epoch that follows are combined to better capture the sleep fragmentation and enhance the detection performance. The spectrogram image was then resized into 224×224 and converted to values between 0 and 1 using normalization. Since the color-image consists of 3 color layers (red, green, and blue), every input matrix became a 3-dimensional matrix (224×224×3). Resized data was entered into a CNN, featuring two layers of two-dimensional convolutions with filters of size 64 and kernel size 12×12. A single convolutional cell (Conv_N) consisted of a convolutional layer, one layer of Batch Normalization, one layer of Rectified Linear Unit (ReLU), one layer of Max pooling and a dropout layer. The filter sizes of two max pooling layers were 8 and 4, respectively and the dropout deactivation rates of two dropout layers were set to 0.5. The data was flattened and followed by one GRU layer (number of hidden units=9) and one fully connected layer and passed through a SoftMax layer and finally outputted the predicted class (either ‘no event’ or ‘apnea event’).

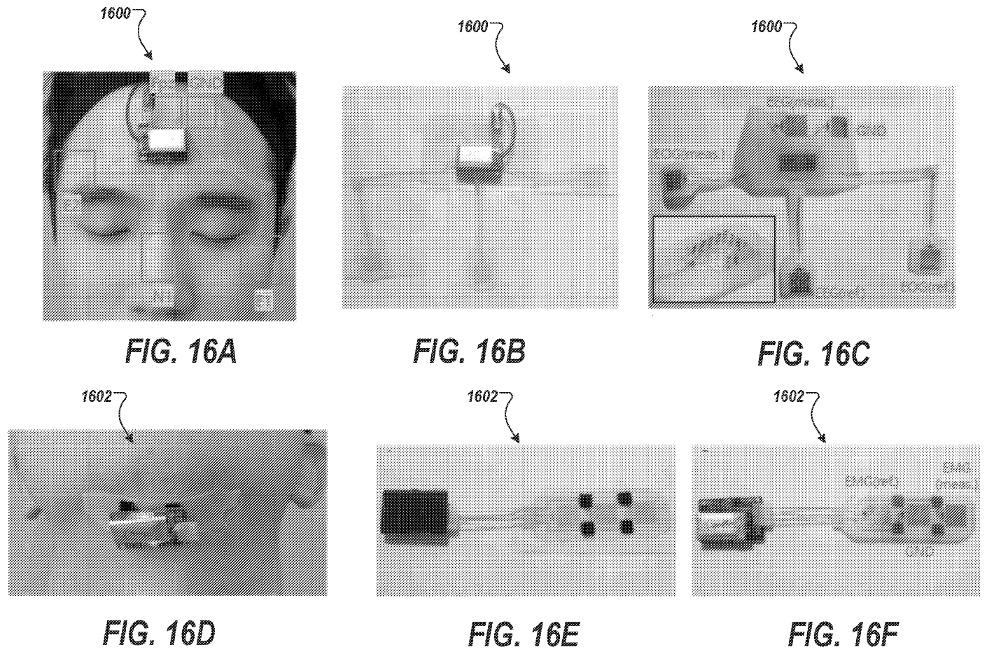

Detailed information of the CNN+GRU architecture is depicted in FIG. 15B, showing a machine learning architecture of a proposed deep neural network for sleep apnea detection. For the training of the CNN+GRU architecture, the cross-entropy loss was used as a loss function, and it was optimized using the ADAM optimizer (learning rate=0.001). Early stopping was used to prevent overfitting by randomly eliminating 20% of the data from the training set and utilizing it as a validation set at the start of the optimization phase. The training was terminated when ten successive decays occurred with no network performance improvement on the validation set and hyper-parameters were optimized by random selection method.

### Comparison to Existing PSG Test

The study compared existing EEG-based wearable sleep monitors to the systems, methods, and devices herein described. Table 4 below summarizes the comparison and captures the unique advantages of a soft wearable sleep patch. A wireless system (e.g., the system herein described) that measures multiple physiological signals offers a clinical assessment of sleep quality and disorder with multiple patients, showing the highest accuracy to date. Specifically, the study showed that the systems of this disclosure had the highest sleep scoring agreement with PSG, in both manual and automated analysis, validating the use of the CNN-based classification model.

TABLE 4
Comparison of EEG-based wearable sleep monitors.

| | | | | Clinical | Manual | Automated | |
|---|---|---|---|---|---|---|---|
| | | | | validation | scoring | scoring | Disorder |
| | | | | w/ | accuracy | accuracy | detection |
| | Form | Electrode | Detecting | patients | (%/ | (%/ | accuracy |
| Ref. | factor | type | signals* | (number) | kappa**) | kappa) | (%) |
| This | Soft, | Stretchable, | EEG, | Yes | 82.43/ | 83.89/ | 88.52*** |
| work | wireless | conformable | EOG, | (8) | 0.74 | 0.76 | |
| | patch | nanomembrane | EMG | | | | |
| | | (dry) | | | | | |
| [12] | Rigid, | Conventional | EEG, | — | — | 74/— | — |
| | wired | gel | EOG, | | | | |
| | headband | (wet) | EMG | | | | |
| [13] | Flexible, | Printed | EEG, | — | — | — | — |
| | wired | composite | EOG, | | | | |
| | patch | (dry) | EMG | | | | |
| [14] | Rigid, | Dry conductive | EEG | — | — | 76.7/ | — |
| | wired | foam and | | | | 0.69 | |
| | headband | conventional | | | | | |
| | | (wet) | | | | | |
| [15] | Rigid, | Conventional | EEG, | Yes | — | 71.3/ | — |
| | wireless | gel | EOG | (40) | | 0.63 | |
| | headband | (wet) | | | | | |
| [16] | Rigid, | Rigid flat metal | EEG | — | — | 83.5/ | — |
| | wireless | (dry) | | | | 0.75 | |
| | headband | | | | | | |
| [17] | Wired ear | Conductive | EEG | — | — | 74.1/ | — |
| | plug | fabric | | | | 0.61 | |
| | | (dry) | | | | | |
| [18] | Wired ear | Rigid iridium | EEG | — | — | 80.5/ | — |
| | plug | oxide | | | | 0.73 | |
| | | (dry) | | | | | |
| [19] | Wired | Printed Ag with | EEG | — | 55/ | 70/ | — |
| | patch | conductive gels | | | 0.38 | 0.58 | |
| | | (wet) | | | | | |

*EEG: electroencephalograms, EOG: electrooculograms, and EMG: electromyograms
**Cohen's kappa value
***Only data showing sleep disorder detection in patients

### Mechanical and Material Characterization of the Soft Wearable System

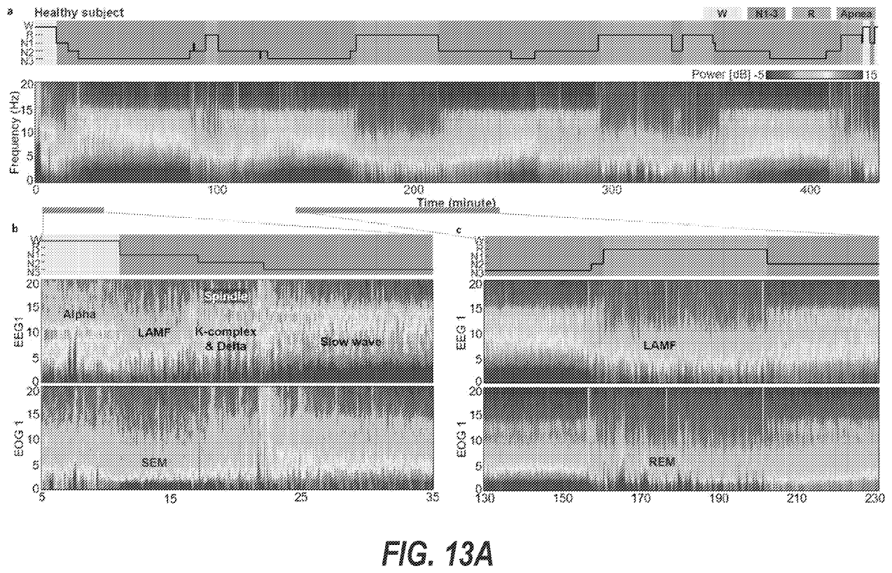

A study was conducted which captures the mechanical properties of the developed soft electrodes and interconnectors (e.g., the serpentine electrodes shown and described in FIGS. 6A-6E) via computational modeling and experimental validation. The finite element analysis (FEA) results in FIGS. 7A and 7B capture the mechanical stretchability of the designed structures up to 30%, showing that maximum strains on the gold and copper membranes remain well below fracture and yield strains (1% and 0.3% respectively)[36, 37]. Additional, the FEA study shows that the electrode and connector can stretch up to 108% and 110% before fracture and yield, respectively, as shown in FIG. 9A. Specifically, FIG. 9A shows stretching FEA up to fracture of gold electrode (subpanel A) and copper connector (subpanel B), each stretching up to the point where the local maximum strain value reaches fracture (1%) and yield (0.3%) respectively.

The maximum tensile strain the structure can endure is above 200% before fractures, as shown in FIGS. 9B-9D. FIGS. 9B-9D provide additional information on mechanical/electrical failure evaluation for the gold electrode, copper connector, and combined system. For example, FIG. 9B shows an image of the combined system with silver paint to allow stable mechanical/electrical connection between the gold electrode and copper connector (the scale bar in the bottom right is 500 micrometers). FIG. 9D (subpanel C) shows that the copper connector failed over 217% strain (the scale bar in the bottom right is 300 micrometers). FIG. 9D illustrates the resistance change of the gold electrode, copper connector, and combined system. The graphs show that the gold electrode failure point (subpanel A) was 249%, the copper connector failure point (subpanel B) was at 214%, and the combined system failure point (subpanel C) was 217%. The copper connector showed lower maximum stretching stability which makes the combined system follow the breakage point of the copper connector.

An experimental study shown in FIG. 7C validates the mechanical reliability of fabricated components during a stretching test. There are no observed fractures or yielding features in the example serpentine electrode stretched to 30%. Cyclic stretching of the electrode and connector and electrical measurements, shown in FIG. 7D, proves the safety for multiple uses. With 1,000 cycles of 30% tensile stretching, there is negligible change in resistance with intermittent artifacts. Experimental validation of the omnidirectional stretchability and elasticity of the fabric shows the fabric's stretchability above 300% with Young's modulus of 1.29 MPa and Poisson's ratio of 0.184, as shown in FIG. 9E. FIG. 9E shows the strain-stress curves of the fabric stretched in three different directions up to failure, showing a nearly uniform result from all three directions.

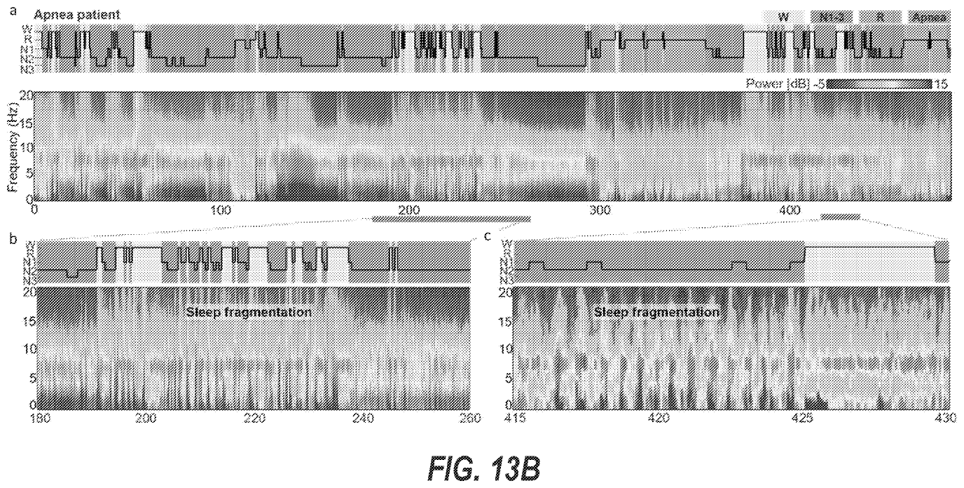

A set of experiments were conducted to find the material's optimal thickness and peeling strength by using the soft silicone adhesive (Silbione). As summarized in FIG. 7E, the membrane's thickness decreases according to the coating speed of the material on a substrate. Experimental conditions and dimensions are also detailed in the array of images in FIG. 10A. For example, FIG. 10A (with subpanels A, B, C, D, E, and F) shows an example of the fabric substrate with thickness 250 μm, thickness of Silbione of 250, 172, 146, 99, 73, and 56 μm with spin coating with 500, 1000, 1500, 2000, and 3000 ROM respectively (scale bars on bottom right are 200 μm). On the other hand, the peeling strength is proportional to the thickness due to the increased energy required for the elastomer's deformation. In this study, it was determined that a 250 μm-thick membrane is ideal for providing enough peeling strength and skin conformability. A graph in FIG. 7F summarizes measured peeling strength values and signal-to-noise ratio (SNR) of measured contact signals with the skin for seven days of sleep. This result shows high degradation of peeling strength when the device is not washed, whereas the soap-washed specimen shows marginally degraded peeling strength. The major degradation factors in

adhesion are dirt and skin oil that can be washed off by cleaning with soap [26]. The SNR values with washed devices show minimal changes in SNR throughout seven days of monitoring, offering a multi-night use of the wearable patch for detecting high-fidelity sleep data at home. When measuring physiological sleep signals, the wearable device's skin contact quality is critical to maintaining low skin-dry electrode contact impedance [28, 30, 38]. Therefore, a model was developed for quantitative analysis by assuming a wearable patch has enough deformability and stretchability to fill the gap between the skin's sinusoidal morphology and the backing layer's flat surface. The skin conformal contact can be determined by this equation showing the interactions with the skin:

$${U}_{conformal} = {U}_{bending} + {U}_{silicone} + {U}_{skin} + {U}_{adhesion} \qquad ( 1 )$$

The model providing this equation is further described below.

### Model of Quantitative Analysis: Derivation of Conformal Contact Model of Electrode and Silicone Elastomer With Stiff Backing Layer on Microstructure of Skin

To make a mechanical model of contact of system on microstructure of skin, the interface between the system and skin is analytically modeled. The microscopic morphology of the skin surface is assumed to be sinusoidal, and modeled as:

$$y ( x ) = \frac{{h}_{rough}}{2} ( 1 + \frac{cos ( 2 π x )}{{λ}_{rough}} ) \qquad ( 2 )$$

In equation (2), h_{rough} is the amplitude, and λ_{rough} is the wavelength of the sinusoidal model of the skin. When the system makes conformal contact with skin, both the system and the skin undergo deformation to create equilibrium sinusoidal morphology with altered amplitude, but with the same wavelength. In this model, the maximum deflection of the electrode, or the new amplitude of the sinusoid, is noted as h. Displacement undergone by the electrode and skin are modeled as:

$$w ( x ) = \frac{h}{2} ( 1 + \frac{cos ( 2 π x )}{{λ}_{rough}} ) \qquad ( 3 )$$

$${u}_{z} ( x ) = y - w = \frac{{h}_{rough} - h}{2} ( 1 + \frac{cos ( 2 π x )}{{λ}_{rough}} ) \qquad ( 4 )$$

In prior works with skin-conformable electronics, the conformality of the system was determined by deriving the interfacial contact energy (U_{conformal}) of the system and skin, which is the sum of three different energies, expressed as:

$${U}_{conformal} = {U}_{bending} + {U}_{skin} + {U}_{adhesion} \qquad ( 5 )$$

In this equation (5), U_{bending}, Uₛₖᵢₙ, and U_{adhesion} represent bending energy of electrode, elastic energy of skin, and contact adhesion energy between the system and skin, respectively. The prior works that used this three-energy model made a major assumption that the entire system behaves as an ultrathin film, and the entire system deforms conformally to that of skin. Bending energy in the three-energy model includes the energy required to bend both the electrode and the supporting silicone elastomer layer. On the other hand, in the example system, it is assumed that the ultrasoft silicone elastomer used, Silbione, undergo normal deformation, rather than bending, to compensate the interface between the flat, rigid surface of the relatively stiff fabric backing layer and the curvilinear, sinusoidal surface of the skin. This new interface is modeled with four-energy as:

$${U}_{conformal} = {U}_{bending} + {U}_{silicone} + {U}_{skin} + {U}_{adhesion} \qquad ( 6 )$$

In this new four-energy interfacial model presented in equation (6), U_{bending} represents the bending energy of the electrode alone, and U_{silicone} represents the elastic deformation energy of the silicone elastomer. In both previous and new model, non-conformal contact is defined as a state where no component experiences any deformation. Therefore, non-conformal contact is defined as a state where the total energy is zero, U_{non-conformal}=0. The electrode bending energy is modeled as:

$${U}_{bending} = \frac{1}{{λ}_{rough}} {\int }_{0}^{{λ}_{rough}} \frac{α {EI}_{electrode} {( {w}^{″} )}^{2}}{2} dx = \frac{α {π}^{4} {EI}_{electrode} {h}^{2}}{{λ}_{rough}^{4}} \qquad ( 7 )$$

In this bending energy model in equation (7), a is areal fraction, or fill-factor, of the mesh-patterned electrode. The effective bending stiffness of the electrode, EI_{electrode}, is determined as:

$${EI}_{electrode} = {\sum }_{i = 1}^{N} {E}_{i} {h}_{i} [ {( b - {\sum }_{j = 1}^{i} {h}_{j} )}^{2} + ( b - {\sum }_{j = 1}^{i} {h}_{j} ) {h}_{i} + \frac{1}{3} {h}_{i}^{2} ] \\ where \qquad ( 8 )$$

$$b = {\sum }_{i = 1}^{N} {E}_{i} {h}_{i} ( {\sum }_{j = 1}^{i} {h}_{j} - \frac{1}{2} {h}_{i} ) / {\sum }_{i = 1}^{n} {E}_{i} {h}_{i} \qquad ( 8 )$$

The effective bending stiffness of the electrode is modeled as a composite of N layers. Eᵢ is the Young's modulus, and hᵢ is the thickness of the i^{th} layer.

The elastic deformation energy of silicone elastomer is modeled as:

$${U}_{silicone} = \frac{1}{{λ}_{rough}} {\int }_{0}^{{λ}_{rough}} \frac{{{E}_{silicone} ( w )}^{2}}{2 {t}_{silicone}^{2}} dx = \frac{{E}_{silicone} {h}^{2}}{16 {t}_{elastomer}^{2}} \qquad ( 9 )$$

In the model presented in equation (9), E_{silicone} and t_{silicone} represent the Young's modulus and thickness of the silicone elastomer, respectively.

Similarly with the previous works, the skin is modeled as a semi-infinite body subject to the surface displacement. The normal stress existing on the surface of the skin is analytically modeled as:

$${σ}_{z} = \frac{π {E}_{skin} ( {h}_{rough} - h )}{2 {λ}_{rough}} cos ( \frac{2 π x}{{λ}_{rough}} ) \qquad ( 10 )$$

Eₛₖᵢₙ represents the Young's modulus of the skin. With the normal stress model in equation (10), the elastic energy of the skin can be derived as:

$${U}_{skin} = \frac{1}{{λ}_{rough}} {\int }_{0}^{{λ}_{rough}} \frac{{σ}_{z} {u}_{z}}{2} dx = \frac{π {{E}_{skin} ( {h}_{rough} - h )}^{2}}{16 {λ}_{rough}} \qquad ( 11 )$$

The interfacial contact adhesion energy can be derived by multiplying work of adhesion (γ) with the contact area. In this case, the contact area of silicone on skin is reduced by the areal fraction. The adhesion energy is modeled as:

$${U}_{adhesion} = - ( 1 - α ) γ {\int }_{0}^{{λ}_{rough}} \sqrt{1 + {( {w}^{′} )}^{2}} dx \approx - ( 1 - α ) γ ( 1 + \frac{{π}^{2} {h}^{2}}{4 {λ}_{rough}^{2}} ) \qquad ( 12 )$$

Through minimization of total energy

$$( \frac{\partial {U}_{conformal}}{\partial h} )$$

with respect to the maximum deflection (h) of electrode and skin, the value of h can be derived as:

$$h = \frac{π {E}_{skin} {h}_{rough}}{\frac{16 {απ}^{4} {EI}_{electrode}}{{λ}_{rough}^{3}} + \frac{{λ}_{rough} {E}_{silicone}}{{t}_{silicone}^{2}} + π {E}_{skin}} \qquad ( 13 )$$

To achieve conformal contact, U_{conformal} of the system should be larger than U_{non-conformal.} In other words, the adhesion energy should be larger than the sum of bending energy of electrode, elastic energy of silicone, and elastic energy of skin. Therefore, the condition to achieve conformal contact can be derived as:

$$( 1 - α ) γ ( 1 + \frac{{π}^{2} {h}^{2}}{4 {λ}_{rough}^{2}} ) > ( \frac{α {π}^{4} {EI}_{electrode} {h}^{2}}{{λ}_{rough}^{4}} + \frac{{E}_{silicone} {h}^{2}}{16 {t}_{elastomer}^{2}} + \frac{π {{E}_{skin} ( {h}_{rough} - h )}^{2}}{16 {λ}_{rough}} ) \qquad ( 14 )$$

In this study, the skin parameter values used are Eₛₖᵢₙ=130 kPa, γ_{rough}=385 μm, and h_{rough}=55 μm. The areal fraction of the electrode a is 0.2687.

### Mechanical and Material Characterization of the Soft Wearable System (Cont.)

In this model, there are three key properties of a patch regarding the elastomeric membrane's thickness work of adhesion and Young's modulus. The experimental study validates the analytical study as summarized in FIGS. 8A-8G. Scanning electron microscope (SEM) images in FIG. 8A and 8B show examples of skin-electrode contact quality, depending on the membrane material and thickness. Among these cases, a soft membrane layer made of Silbione (500 RPM; thickness: 250 μm; FIG. 8A) offers the best contact quality, which is more advantageous than the one in FIG. 8B (thickness: 56 μm) due to less amount of strain required for conformal deformation.

A graph in FIG. 8E shows an analytical calculation of total interface energy with varying thicknesses of Silbione, where more negative energy from thicker membranes indicates higher conformability. On the other hand, two other examples of Ecoflex30 (FIG. 8C) and PDMS (FIG. 8D) have poor contact quality due to the high Young's modulus and low work of adhesion. A diagram in FIG. 8F summarizes the analytical study outcomes of this study, showing the relationship between work of adhesion, modulus, and resultant conformal skin contact. FIG. 10B shows details of the experimental setup and measured values of the work of adhesion for each membrane, including a photo of a test setup. FIG. 10B also shows curve fit lines and their work of adhesion values for three materials: Silbione, Ecoflex 00-30, and PDMS.

Finally, the study measured skin-electrode contact impedance data to compare the performance of the wearable patches with different membrane substrates with the standard Ag/AgCl wet electrode. As summarized in FIG. 8G, the soft adhesive membrane in FIG. 8A shows the best skin-contact quality, like the gel electrode.

### Clinical Study Outcomes of Sleep Quality via Comparison With the Gold-Standard PSG

This study demonstrates the fabricated wearable sleep device's performance via side-to-side comparison with the gold-standard PSG setup. A photo in FIG. 11A shows a subject in a sleep clinic wearing two different systems; there are two unobtrusive wearable patches on the forehead and chin, but the PSG setup requires over fifteen wired bulky sensors and a separate data acquisition system. The physiological signals in FIG. 11B, measured during sleep, compares the data quality in detecting five sleep stages (awake, N1, N2, N3, and REM); PSG and wearable patch data come from locations of F3-M2 channel and EEG1 channel, respectively. The visualization and morphology analysis of these signals is critical because the standard sleep analysis uses this method [31]. Overall, the characteristic signal morphology from the wearable patch shows high similarity to that from PSG, and the patch's data can clearly distinguish each sleep/wake stage. The average SNR value of the wearable patch system (22.77 dB) is comparable to that of PSG (25.52 dB). The lower signal amplitude comes from the patch's reference location on the nose, which is an acceptable alternative place [39].

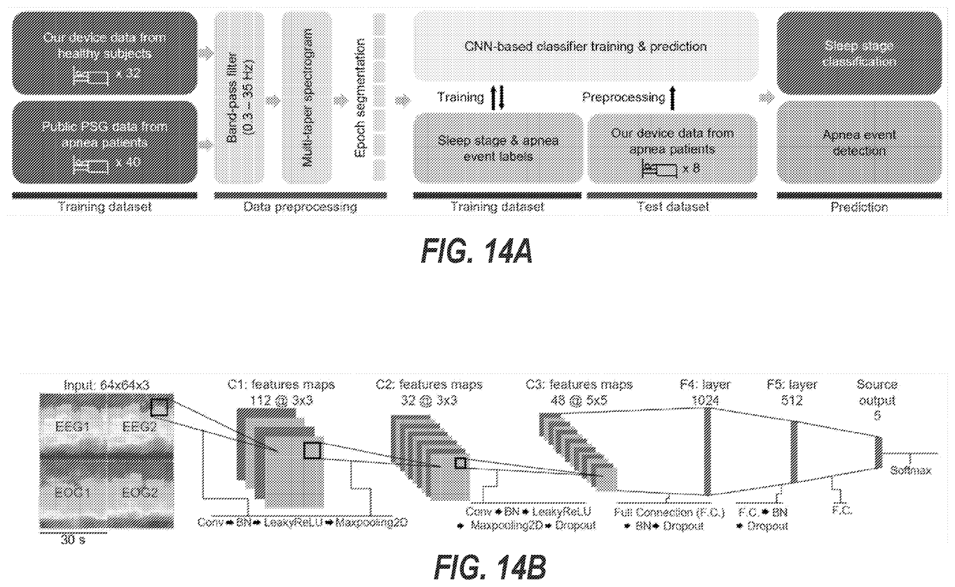

Raw sleep data measured by the wearable patch is shown in FIG. 12A (having subpanels A, B, C, D, and E) with all five channels during five sleep stages. 10-second samples are plotted, showing raw signals measured from all five channels of the devices (Chin EMG, EOG2, EOG1, EEG2, and EEG1), capturing characteristic signals of each of the five sleep stages: (a) Awake, (b) Non-REM 1, (c) Non-REM 2, (d) Non-REM 3, and (e) REM. The Y-axis unit is in microvolt (μV).

Additional analysis in FIG. 11C-11D compares spectrogram sleep data, showing similar spectral profiles throughout the sleep. The spectral power comparison over the four main EEG frequency bands (delta, theta, alpha, and sigma) shows high correspondence with the Pearson correlation value of 0.76 and p-value of less than 10⁻¹⁰.

Details of the power values appear in FIG. 12B, showing spectral power comparison of EEG signals from PSG and the example system of this disclosure. The plots compare spectral powers of F3-M2 channel of PSG and EEG 1 channel of the device simultaneously measured from the same subject over the four main EEG frequency bands: (a) alpha, (b) theta, (c) delta, and (d) sigma. The signal quality was further validated by scoring the sleep data from both systems. A sleep technician conducted this scoring with blind data sets to avoid bias.

Summarized results in FIG. 11E-11F present two representative examples of the manual scoring analysis, which shows strong agreement (87.50% and 88.19%) with a high Cohen's kappa value (0.80 and 0.82) between the two systems. The confusion matrix in FIG. 11G summarizes the comparison results acquired from eight subjects detecting five sleep stages. The overall accuracy is 82.43% with Cohen's kappa value of 0.74, which is very close to the reported

average interscorer reliability value of 82.0% with Cohen's kappa value of 0.76 [40]. More importantly, the wearable patch shows the highest performance in scoring accuracy among wearable EEG monitors (details in Table 1). The accuracy is calculated by following this equation: Accuracy=(true positive+true negative)/(true positive+false positive+true negative+false negative).

Overall, this study validates that the wearable patch can measure physiological sleep data at the level of clinical standards. More importantly, compared to the standard PSG system, the wearable patch disclosed herein has unique advantages of portability, accessibility, and multi-day use. This device can be readily usable at home for detecting sleep data for a week. An example in FIG. 12C captures 7-day-long sleep data measured at home while showing the maintained data quality throughout the measurements. Specifically, FIG. 12C shows spectrogram data of seven consecutive sleep measurements generated from EEG1 signals at each day of seven consecutive sleep measurements with the example system. Data was taken during (a) the first week and (b) the second week of the whole measurement, showing a negligible change in the signal strengths and the calculated SNR values of the delta wave during N3 changes.

### Measurements of Sleep Stages and Detection of Sleep Apnea From Controls and Patients

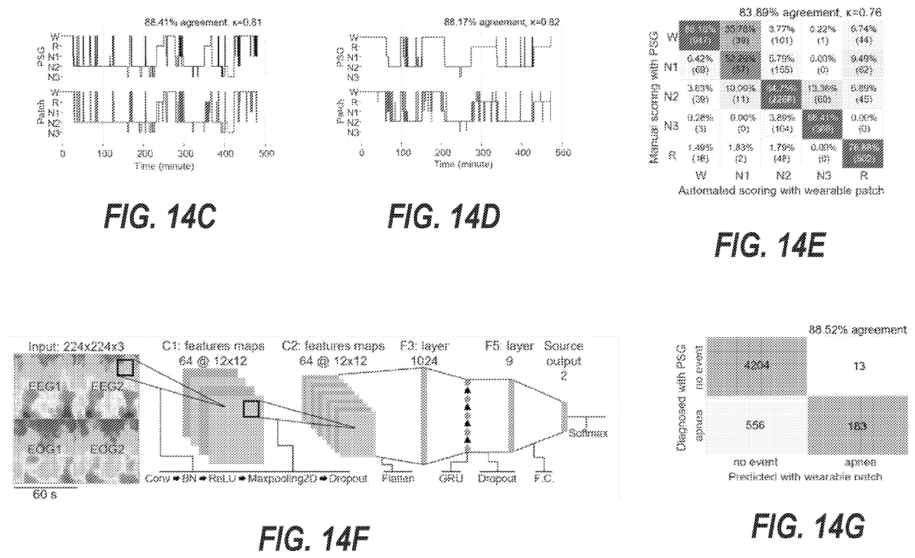

FIG. 13A (having subpanels A, B, and C) shows an EEG hypnogram and a spectrogram measured from a healthy control using the wireless wearable patch. As clearly categorized in the hypnogram, this subject shows stable and uniform cycles of sleep stages without the indication of apnea. In this analysis, the multi-taper spectrogram analysis is used for quantifying various features of EEG and EOG in both time and frequency domains, providing a more accurate analysis than the traditional single-taper spectrogram [41].

A representative example in FIG. 13C shows how the multi-taper method was used to analyze sleep data. Specifically, FIG. 13C shows comparison of single-and multi-taper spectrogram data. Plots are shown comparing (a) band pass-filtered raw EEG 1 signals (in subpanel A), (b) standard single-taper spectrogram (in subpanel B), and (c) multi-taper spectrogram that features higher clarity with minimized bias and variance in spectral estimation (in subpanel C).

A set of data in lower portion of FIG. 13A (subpanel B) shows a close-up view of spectrograms and EEG and EOG that capture the progression from the awake stage (W) to non-rapid eye movement (N3) stage with corresponding characteristic signal features. During stage W, eyes are closed, showing 10 Hz alpha waves as indicated in the EEG spectrogram. During N1, low-amplitude mixed-frequency (LAMF) signals are observed, and its spectrogram shows weaker spectral power than other stages. During N2, the EEG spectrogram captures the increase in low-frequency delta wave activity, with frequent occurrence of microevents such as K-complex, delta waves, and sleep spindle. During N3, EEG spectrogram depicts the continuous and uniform activity of strong low-frequency delta (slow wave). Comparison of spectrograms of EOG and EEG further facilitates distinguishing of sleep stages. During stage N1, spectrogram of EOG shows a strong activity in the lower frequency that corresponds to the slow eye movement (SEM). Besides N1, EOG spectrogram shows a mirror-image of EEG spectrogram with weaker spectral power. Additional datasets in subpanel C of FIG. 13A present other features. During stage R, the EEG spectrogram shows LAMF, similarly with N1, while EOG spectrogram captures strong and irregular activity in the lower frequency that corresponds to rapid eye movement (REM).

In contrast to the healthy subject, a patient with severe sleep apnea shows completely different, highly fragmented sleep cycles with frequent arousals due to apnea events, causing deteriorated quality of sleep and tiredness. Data from a patient with sleep apnea is shown in FIG. 13B. The hypnogram in FIG. 13B captures the numerous arousals following series of apnea, and the corresponding spectrogram is highly fragmented and dominated with alpha wave around the arousal events. Spectrogram data in subpanels B and C of FIG. 13B further depicts the characteristics of fragmented sleep signals. At the onset of each apnea event, the low-frequency spectral power typical of non-REM sleep becomes suppressed, and a burst of spectral power throughout a wide range of frequency, from delta to sigma, occurs after the apnea event. Overall, this study shows the capability of the example data analysis to distinguish the difference between healthy sleep and apnea.

### Automated Sleep Scoring and Quantitative Diagnosis of Sleep Apnea Using CNN

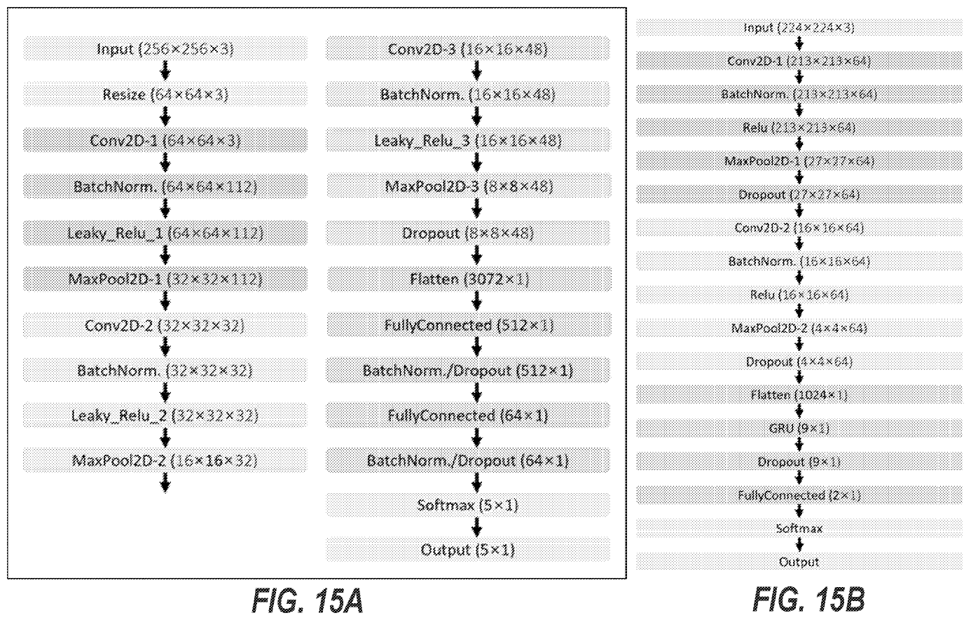

The standard method in sleep analysis is to use visual data observation and manual scoring by technicians, causing delay, additional costs, and human errors [5, 42, 43]. Thus, the automated sleep classification and scoring methods described herein use deep learning CNN, as shown in FIGS. 14A-14G. FIG. 14A depicts an entire flow from data acquisition to training, processing, and prediction. The illustration in FIG. 14B shows the deep learning architecture developed in a study that uses sleep data measured by the wearable patch for automated sleep scoring. Details of the CNN algorithm, data processing, and classification processes for sleep stage detection and sleep apnea detection also appear in FIGS. 15A and 15B as well as Tables 1 and 2.

Graphs in FIG. 14C and 14D show two representative results of the automated sleep scoring. When the automated scoring data from the wearable patch is compared to that from the PSG scoring case, there is strong agreement between two methods with an accuracy of 88.41% and 88.17% and Cohen's kappa values of 0.81 and 0.82. The correlation outcomes of sleep scoring have similar results to the manual scoring datasets from FIGS. 11E-11F. The confusion matrix in FIG. 14E summarizes the performance of the example CNN algorithm compared to manual scoring with PSG. The overall agreement and Cohen's kappa between the automated and manual scoring (83.89% and 0.76), showing a better performance than the reported average interscorer reliability (82.0%) [40].

A set of diagrams in FIG. 11F depicts the input data and the CNN architecture for sleep apnea detection. In the analysis of the confusion matrix (FIG. 11G), the system classified 4760 epochs as no event and 196 epochs as apnea. Among them, a sleep technician diagnosed 4204 epochs as normal and 556 epochs as apnea in the PSG data. Out of the 196 epochs, 183 epochs were diagnosed as apnea while 13 epochs (6.63%) were diagnosed as normal. Thus, overall, with eight patients, the system's performance shows high accuracy of 88.52%, demonstrating the first study of detecting sleep disorder with the wearable-machine learning system. Collectively, this study shows a potential of the system of this disclosure to offer real-time, automated, and accurate detection of sleep stages and disorders, which will advance portable sleep monitoring and home healthcare.

### Example System and Prototype

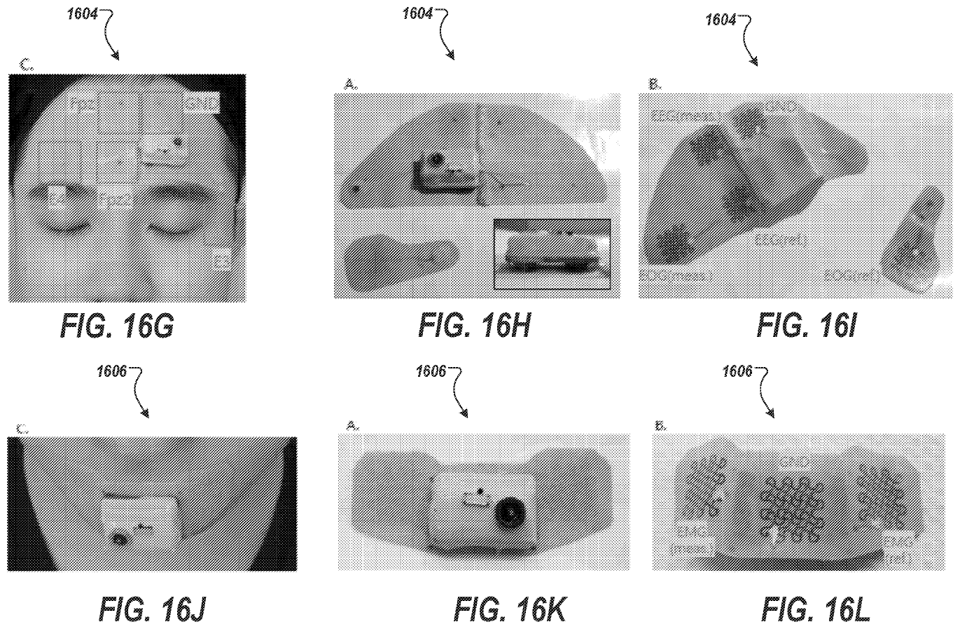

A further example system and prototype device is shown in FIGS. 16A-16L and herein described. For example, the system and prototype devices 1600, 1602, 1604, and 1606 of FIGS. 16A-16L was fabricated and tested in alignment with the above-described fabrication methods and experimental results. Similar to the device 100 of FIG. 1, the devices 1600, 1602, 1604, and 1606 include gold membrane electrodes, hypoallergenic silicone adhesive substrate, and custom PCB for wireless data transmission.

FIG. 16A shows an example device 1600 disposed on the face of a user. FIGS. 16B and 16C show the front and back sides of the device 1600 including the general structure of the substrate, electrodes, wireless PCB, and connectors. The device 1600 is configured to detect, transmit, and/or analyze EEG and/or EOG signals.

FIG. 16D shows an example device 1602, similar to device 1600, disposed on the chin of a user. FIGS. 16E and 16F show the front and back sides of the device 1602 including the general structure of the substrate, electrodes, wireless PCB, and connectors. The device 1602 is configured to detect, transmit, and/or analyze EMG signals.

FIG. 16G shows an example device 1604, similar to device 1600, disposed on the face of a user. FIGS. 16H and 16I shown the front and back sides of the device 1604 including the general structure of the substrate, electrodes, wireless PCB, and connectors.

FIG. 16J shows an example device 1606, similar to device 1604, disposed on the chin of a user. FIGS. 16K and 16L show the front and back sides of the device 1606 including the general structure of the substrate, electrodes, wireless PCB, and connectors.

### Discussion

Sleep is an integral part of the human life cycle, and its quality and duration play a critical role in physical, mental, and social health [1-4]. As an increasing number of people recognize sleep as one of the essential factors in a healthy lifestyle, there has been rapid growth in sleep studies. The market size of the global sleep-related economy was $432 billion and is expected to grow to $585 billion by 2024 [5]. Despite this elevated awareness, the average quality of a person's sleep has declined, and sleep disorder has become increasingly prevalent. Insufficient and poor sleep cause reduced labor productivity and increased mortality rates, leading to an economic loss of $411 billion in 2015, which is expected to grow to $467 billion in 2030 [6]. Moreover, the American Association of Sleep Medicine (AASM) reports that one of the most prevalent sleep disorders, obstructive sleep apnea, afflicted 12% of the adult population in the United States and noted that 80% of them were left undiagnosed [7]. The gold standard of sleep monitoring is polysomnography (PSG), but its various downsides impede its accessibility [8]. PSG involves comprehensive measurements of patient physiological signals, including electroencephalograms (EEG), electrooculograms (EOG), electromyograms (EMG), pulse oximetry, and more. Due to its complexity and difficulty in both hardware setups and data analysis, standard PSG is conducted at a specialized hospital with a certified sleep technologist, resulting in a time and cost burden. Moreover, due to the foreign environment with numerous hard-wired sensors and electronics placed throughout the body, patients may not have their natural sleep patterns, which can lead to inaccurate sleep quality assessment and disorder diagnosis.

Recent progress in the developments of wearable devices has presented alternative ways for sleep monitoring at home [8, 9]. One of the widely used platforms is a wristband with integrated photoplethysmography and motion sensors. The convenience of the watch is attractive but fails to comprehensively cover the amount of physiological information needed for precise in-depth sleep analysis [10]. EEG is often the most direct indicator of sleep stages and sleep disorders [11]. Some headband devices can measure EEG and provide more precise sleep analysis [12-16]. However, their bulky and rigid form factor discourages users from mounting them on their heads during sleep. Recent studies presented alternative form factors for sleep EEG measurement, such as those mounted either in or around the ear [17-19]. Despite the improved usability, the alternative device locations suffer from a poor signal quality and less accurate sleep analysis. For comfortable and seamless integration with a user's body for high-quality physiological monitoring, many recent works on developing soft wearable systems have been directed toward enhancing various aspects of epidermal electronics [20-21]. Prior wearable devices show enhanced skin-contact quality by integrating the system on a soft substrate [22-25]. However, the required membrane materials for such ultrathin devices limit the number of sensors that can be embedded in the system while causing mechanical reliability issues.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “approximately” one particular value and/or to “about” or “approximately” another particular value. When such a range is expressed, other exemplary embodiments include the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.

### References

[1] Buysse, D. J. Sleep health: can we define it? Does it matter? Sleep 37, 9-17 (2014).
[2] Gulia, K. K. & Kumar, V. M. Importance of sleep for health and wellbeing amidst COVID-19 pandemic. Sleep and vigilance 4, 49-50 (2020).
[3] Hublin, C., Partinen, M., Koskenvuo, M. & Kaprio, J. Sleep and mortality: a population-based 22-year follow-up study. Sleep 30, 1245-1253 (2007).
[4] Patel, S. R. et al. A prospective study of sleep duration and mortality risk in women. Sleep 27, 440-444 (2004).
[5] Kwon, S., Kim, H. & Yeo, W.-H. Recent advances in wearable sensors and portable electronics for sleep monitoring. Iscience 24, 102461 (2021).
[6] Hafner, M., Stepanek, M., Taylor, J., Troxel, W. M. & Van Stolk, C. Why sleep matters—the economic costs of insufficient sleep: a cross-country comparative analysis. Rand health quarterly 6 (2017).
[7] Watson, N. F. Health care savings: the economic value of diagnostic and therapeutic care for obstructive sleep apnea. Journal of Clinical Sleep Medicine 12, 1075-1077 (2016).
[8] Ibáñez, V., Silva, J., Navarro, E. & Cauli, O. Sleep assessment devices: types, market analysis, and a critical view on accuracy and validation. Expert Review of Medical Devices 16, 1041-1052 (2019).
[9] Guillodo, E. et al. Clinical applications of mobile health wearable-based sleep monitoring: systematic review. JMIR mHealth and uHealth 8, e10733 (2020).
[10] Pan, Q., Brulin, D. & Campo, E. Current status and future challenges of sleep monitoring systems: systematic review. JMIR Biomedical Engineering 5, e20921 (2020).
[11] Berry, R. B. et al. Vol. 13 665-666 (American Academy of Sleep Medicine, 2017).
[12] Arnal, P. J. et al. The Dreem headband as an alternative to polysomnography for EEG signal acquisition and sleep staging. BioRxiv, 662734 (2019).
[13] Kim, S.-W. et al. Wearable multi-biosignal analysis integrated interface with direct sleep-stage classification. IEEE Access 8, 46131-46140 (2020).
[14] Levendowski, D. J. et al. The accuracy, night-to-night variability, and stability of frontopolar sleep electroencephalography biomarkers. Journal of Clinical Sleep Medicine 13, 791-803 (2017).
[15] Lin, C.-T. et al. Forehead EEG in support of future feasible personal healthcare solutions: Sleep management, headache prevention, and depression treatment. IEEE Access 5, 10612-10621 (2017).
[16] Shustak, S. et al. Home monitoring of sleep with a temporary-tattoo EEG, EOG and EMG electrode array: a feasibility study. Journal of neural engineering 16, 026024 (2019).
[17] Mikkelsen, K. B. et al. Accurate whole-night sleep monitoring with dry-contact ear-EEG. Scientific reports 9, 1-12 (2019).
[18] Nakamura, T., Alqurashi, Y. D., Morrell, M. J. & Mandic, D. P. Hearables: automatic overnight sleep monitoring with standardized in-ear EEG sensor. IEEE Transactions on Biomedical Engineering 67, 203-212 (2019).
[19] Sterr, A. et al. Sleep EEG derived from behind-the-ear electrodes (CEEGrid) compared to standard polysomnography: a proof of concept study. Frontiers in human neuroscience 12, 452 (2018).
[20] Kim, D.-H. et al. Epidermal electronics. science 333, 838-843 (2011).
[21] Lim, H. R. et al. Advanced soft materials, sensor integrations, and applications of wearable flexible hybrid electronics in healthcare, energy, and environment. Advanced Materials 32, 1901924 (2020).
[22] Kim, H. et al. Fully Integrated, Stretchable, Wireless Skin-Conformal Bioelectronics for Continuous Stress Monitoring in Daily Life. Advanced Science 7, 2000810 (2020).
[23] Kim, Y. S. et al. Soft Wireless Bioelectronics Designed for Real-Time, Continuous Health Monitoring of Farmworkers. Advanced Healthcare Materials, 2200170 (2022).
[24] Kim, Y. S. et al. All-in-one, wireless, stretchable hybrid electronics for smart, connected, and ambulatory physiological monitoring. Advanced Science 6, 1900939 (2019).
[25] Kim, Y.-S., Mahmood, M., Kwon, S., Herbert, R. & Yeo, W.-H. in Nano-, Bio-, Info-Tech Sensors and 3D Systems III. 1096903 (SPIE).
[26] Jang, K.-I. et al. Rugged and breathable forms of stretchable electronics with adherent composite substrates for transcutaneous monitoring. Nature communications 5, 1-10 (2014).
[27] Kwon, Y.-T. et al. Breathable, large-area epidermal electronic systems for recording electromyographic activity during operant conditioning of H-reflex. Biosensors and Bioelectronics 165, 112404 (2020).
[28] Jeong, J. W. et al. Materials and optimized designs for human-machine interfaces via epidermal electronics. Advanced Materials 25, 6839-6846 (2013).
[29] Mishra, S. et al. Soft, conformal bioelectronics for a wireless human-wheelchair interface. Biosensors and Bioelectronics 91, 796-803 (2017).
[30] Yeo, W. H. et al. Multifunctional epidermal electronics printed directly onto the skin. Advanced materials 25, 2773-2778 (2013).
[31] Berry, R. B. et al. The AASM manual for the scoring of sleep and associated events. Rules, Terminology and Technical Specifications, Darien, Illinois, American Academy of Sleep Medicine 176, 2012 (2012).
[32] Fan, J. A. et al. Fractal design concepts for stretchable electronics. Nature communications 5, 1-8 (2014).
[33] Lee, K. et al. Mechano-acoustic sensing of physiological processes and body motions via a soft wireless device placed at the suprasternal notch. Nature biomedical engineering 4, 148-158 (2020).
[34] Rodeheaver, N., Kim, H., Herbert, R., Seo, H. & Yeo, W.-H. Breathable, Wireless, Thin-Film Wearable Biopatch Using Noise-Reduction Mechanisms. ACS Applied Electronic Materials 4, 503-512 (2022).
[35] Rodeheaver, N. et al. Strain-Isolating Materials and Interfacial Physics for Soft Wearable Bioelectronics and Wireless, Motion Artifact-Controlled Health Monitoring. Advanced Functional Materials 31, 2104070 (2021).
[36] Kwon, S. et al. Skin-conformal, soft material-enabled bioelectronic system with minimized motion artifacts for reliable health and performance monitoring of athletes. Biosensors and Bioelectronics 151, 111981 (2020).
[37] Mahmood, M. et al. Soft nanomembrane sensors and flexible hybrid bioelectronics for wireless quantification of blepharospasm. IEEE Transactions on Biomedical Engineering 67, 3094-3100 (2020).
[38] Norton, J. J. et al. Soft, curved electrode systems capable of integration on the auricle as a persistent brain-computer interface. Proceedings of the National Academy of Sciences 112, 3920-3925 (2015).
[39] Yao, D. et al. Which reference should we use for EEG and ERP practice? Brain topography 32, 530-549 (2019).
[40] Danker-hopfe, H. et al. Interrater reliability for sleep scoring according to the Rechtschaffen & Kales and the new AASM standard. Journal of sleep research 18, 74-84 (2009).
[41] Prerau, M. J., Brown, R. E., Bianchi, M. T., Ellenbogen, J. M. & Purdon, P. L. Sleep neurophysiological dynamics through the lens of multitaper spectral analysis. Physiology 32, 60-92 (2017).
[42] Zavanelli, N. et al. At-home wireless monitoring of acute hemodynamic disturbances to detect sleep apnea and sleep stages via a soft sternal patch. Science advances 7, eab14146 (2021).
[43] Kwon, K., Kwon, S. & Yeo, W.-H. Automatic and Accurate Sleep Stage Classification via a Convolutional Deep Neural Network and Nanomembrane Electrodes. Biosensors 12, 155 (2022).

Listing of claims:

1. A wearable biopatch device configured for sleep monitoring, the device comprising:
a hypoallergenic silicone adhesive substrate comprising an inner surface and an outer surface;
an integrated sensor system coupled to the inner surface of the silicone adhesive substrate, the integrated sensor system comprising a plurality of electrodes placed in predetermined locations and electrical connections between the plurality of electrodes, including at least three electrodes configured for EEG sensing and at least three electrodes for EOG sensing;
a silicone fabric substrate disposed on the outer surface of the silicone adhesive substrate, the hypoallergenic silicone adhesive substrate and silicone fabric substrate providing conformal and stretchable substrate for conformal and stretchable contact on a facial area; and
at least one circuit configured for wireless data transmission disposed on a portion of the silicone fabric substrate, the at least one circuit comprising thin, flexible film connectors.

2. The device of claim 1, wherein the wearable biopatch device is attachable a forehead portion of the facial area and configured to measure EEG and EOG,
the device further comprising a second wearable biopatch attachable to the chin and configured to measure EMG, the second wearable biopatch being electrically linked to the wearable biopatch device to provide synchronous measurements of the EEG sensing, EOG sensing, and EMG sensing.

3. The device of claim 1, wherein the device is configured to monitor brain activity and eye movement to provide the EEG sensing and EOG sensing to an analysis system configured with a trained machine learning or neural network to provide sleep stage classification and apnea event detection, wherein the trained machine learning or neural network is configured to provide output for monitoring, tracking, and/or diagnose obstructive sleep apnea.

4. The device of claim 1, wherein the device is configured to monitor brain activity, eye movement, and facial muscle activity signals EEG sensing, EOG sensing, and EMG sensing to an analysis system configured with a trained machine learning or neural network to provide sleep stage classification and apnea event detection, wherein the trained machine learning or neural network is configured to provide output for monitoring, tracking, and/or diagnose obstructive sleep apnea.

5. The device of claim 1, wherein a portion of the plurality of electrodes are stretchable and formed in a meandering or serpentine structure.

6. The device of claim 1, wherein all of the plurality of electrodes are stretchable and formed in a meandering or serpentine structure.

7. The device of claim 1, wherein the at least three electrodes configured for EEG sensing includes a common ground electrode, a common reference electrode, and a first recording electrode, and wherein the at least three electrodes configured for EOG sensing includes a second recording electrode, the common ground electrode, and the common reference electrode.

8. The device of claim 1, wherein the at least one circuit further comprises: a multi-channel differential amplifier; a Bluetooth-low-energy microcontroller; and an antenna.

9. The device of claim 1, wherein the silicone fabric substrate comprises polytetrafluoroethylene (PTFE).

10. The device of claim 1, wherein the hypoallergenic silicone adhesive substrate is between 50 micrometers and 300 micrometers thick.

11. The device of claim 1, further comprising a chest-mounted cardiorespiratory patch.

12. The device of claim 1 further comprising:
a remote analysis system separate from the wearable biopatch device, the remote analysis system being configured to receive signals from the wearable biopatch device and either (i) relay the received signals to a cloud analysis system having a trained machine learning or neural network or (ii) perform the analysis with a locally executing trained machine learning or neural network.

13. The device of claim 12, wherein the remote analysis system or the cloud analysis system is configured to provide a sleep score associated with a sleep stage classification and apnea event detection.

14. A system comprising:
the wearable biopatch device of claim 1; and
an analysis system separate from the wearable biopatch device, the remote analysis system being configured to receive signals from the wearable biopatch device and either (i) relay the received signals to a cloud analysis system having a trained machine learning or neural network or (ii) perform the analysis with a locally executing trained machine learning or neural network.

15. The system of claim 14, wherein the analysis system comprises cloud infrastructure.

16. The system of claim 14, wherein the analysis system comprises a smart phone, a tablet, or any other personal computing device having a user interface configured to display at least one of the transmitted data and the sleep score and configured with a short-range communication interface.

17. A method of sleep monitoring comprising:
providing a wearable biopatch device comprising:
a hypoallergenic silicone adhesive substrate comprising an inner surface and an outer surface;
an integrated sensor system coupled to the inner surface of the silicone adhesive substrate, the integrated sensor system comprising a plurality of electrodes placed in predetermined locations and electrical connections between the plurality of electrodes, including at least three electrodes configured for EEG sensing and at least three electrodes for EOG sensing;
a silicone fabric substrate disposed on the outer surface of the silicone adhesive substrate, the hypoallergenic silicone adhesive substrate and silicone fabric substrate providing conformal and stretchable substrate for conformal and stretchable contact on a facial area; and
at least one circuit configured for wireless data transmission disposed on a portion of the silicone fabric substrate, the at least one circuit comprising thin, flexible film connectors;
sensing, via the plurality of electrodes, EEG signals from the at least three electrodes configured for EEG sensing and EOG signals from the at least three electrodes configured for EOG sensing over a period spanning at least one REM cycle;
transmitting the sensed EEG signals and the sensed EOG signals to local computing device;
processing and segmenting, at the local computing device, the sensed EEG signals and the sensed EOG signals;
analyzing, via the local computing device or a remote analysis system that received the data from the local computing device, the segmented EOG signals and the segment EEG signals via a trained machine learning operation; and
providing a sleep score associated with a sleep disorder to a graphical user interface based on the analyzed brain activity data.

18. The method of claim 17, further comprising:
classifying, based on the sleep score, a sleep stage; and
detecting, based on the sleep score, a sleep apnea event.

19. The method of claim 17, wherein the at least three electrodes associated with the EEG sensing are placed on the forehead to acquire the EEG signals, and wherein the at least three electrodes associated with the EOG sensing are placed at the temple to acquire the EOG signals.

20. The method of claim 17, wherein the wearable biopatch device is self-applying to be installed by the subject, and wherein the EEG signals and EOG signals are monitored by a user at a remote location.
